(19) United States
(12) Patent Application Publication
(10) Pub. No.: US 2008/0286218 A1
(43) Pub. Date: Nov. 20, 2008

(54) Cosmetic Compositions Containing at Least One Conditioning Agent and at Least One Ethylene Polymer with Polyethylene Glycol Grafts

(76) Inventors: Franck Giroud, Clichy (FR); Laurence Paul, Saint Leu La Foret (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/631,118

(22) PCT Filed: Jul. 1, 2005
(86) PCT No.: PCT/FR05/01702

§ 371 Date: Sep. 11, 2007

Related U.S. Application Data
(60) Provisional application No. 60/586,728, filed on Jul. 12, 2004.

(30) Foreign Application Priority Data
Jul. 2, 2004 (FR) 0451410

(51) Int. Cl. A61K 8/81; A61Q 19/00; A61Q 5/00; A61Q 3/00

(52) U.S. Cl. 424/61; 424/70.11; 424/70.12; 424/70.13; 424/70.2; 424/78.03

(57) ABSTRACT

The invention concerns cosmetic compositions comprising: A) at least one conditioning agent selected among synthetic oils, mineral oils, vegetable oils, fluorinated and perfluorinated oils, natural or synthetic waxes, silicones, cationic polymers whereof the cationic filler density is less than 5 meq/g, cationic proteins, cationic protein hydrolysates, ceramide type compounds, cationic surfactants, fatty amines, fatty acids and their derivatives, as well as mixtures of those various compounds, and B) at least one ethylene copolymer comprising, in weight percentage based on the total weight of the polymer: a) 10-60 wt. % of one or more monomers of formula (I) as defined below; b) 40-90 wt. % of a substantially cationic monomer selected among (i) one or more cationic monomers of formula (IIa); (ii) one or more amphoteric monomers of formulae (IIc) and (IId); and (iii) a mixture of one or more cationic monomers of formula (IIa) with one or more anionic monomers selected among maleic anhydride and/or those of formula (IIb); and/or with one or more amphoteric monomers selected among those of formulae (IIc) and (IId), c) an optionally 0-50 wt. % of nonionic hydrophilic monomers, excluding methyl acrylate, methyl methacrylate and isopropyl acrylate when they are present in an amount not less than 10 wt. %. Hair treated with said compositions are more easily disentangled during rinsing, and exhibits softness after drying. The inventive compositions also allow particularly interesting hair brushing after drying.

The present invention relates to compositions comprising at least one conditioning agent and at least one ethylenic copolymer with polyethylene glycol grafts.

It is known practice to use polymers in cosmetics, and especially in haircare in leave-in products, for example to give the hair hold or styling.

In the field of “rinse-out” compositions, such as shampoos or hair conditioners, water-soluble synthetic cationic polymers are also used, which are known to give the hair a good cosmeticity; however, these polymers provide no hair shaping effect. This is likewise the case for cationic nature-derived polymers such as modified guar gums, which also provide a cosmetic nature without allowing shaping. In the field of rinse-out compositions, polymers do not provide sufficient styling associated with an acceptable cosmeticity.

The aim of the present invention is to provide cosmetic compositions comprising polymers capable of providing a real styling effect while at the same time maintaining an acceptable cosmeticity for the compositions, and especially sparingly viscous polymers that only slightly modify the viscosity of the compositions comprising them.

After considerable research, the Applicant has discovered that the use of polymers comprising, inter alia, monomers of the polyethylene glycol (meth)acrylate type as defined below can allow the preparation of rinse-out or leave-in styling compositions of adequate cosmeticity.

Polymers containing polyethylene glycol(meth)acrylate (MPEG) units are described in the prior art.

Thus, EP 372 546 discloses copolymers based on MPEG and monomers of C1-C8 alkyl(meth)acrylamide type, which may comprise cationic monomers. However, these polymers comprise only a small proportion of cationic monomers, which does not allow them to generate adequate cosmetic effects, especially deposition on the hair that is sufficient to provide the desired properties.

Document JP2002-322 219 describes polymers containing MPEG units in combination with hydrophobic monomers based on polypropylene glycol (PPO) or polytetramethylene oxide, and cationic monomers. However, it has been found that these polymers comprising hydrophobic monomers do not allow satisfactory cosmetic properties to be obtained.

A composition comprising cationic polymers in which the monomers of PEG type are combined with monomers comprising quaternary amine units is also known from patent JP2002-284 627. However, the presence of quaternary units may induce, gradually in the course of applications, extra deposition that may, in certain cases, harm the cosmetic quality of the composition. Moreover, these polymers contain, a low content of cationic charge, of about 0.5% to 6%, which does not allow optimum affinity for the hair.

Document JP2000-302 649 describes a haircare composition comprising a polymer that comprises cationic or amphoteric monomers, monomers with a polyether group, especially of PEG or PPO type, and also optional monomers that may be mainly hydrophobic (for example stearyl methacrylate).

Haircare compositions comprising a polymer that comprises monomers of MPEG type in combination with ionic, cationic or amphoteric monomers, and additional monomers of C1-C24 alkyl meth)acrylate type, which are mainly hydrophobic, are also known from patent JP07-285 831. However, the presence of hydrophobic comonomers, for example of butyl or stearyl acrylate type, does not make it possible to obtain adequate cosmetic properties, and especially does not make it possible to obtain good disentangling of wet hair, just after shampooing.

Patent application WO 03/075 867 is also known, which describes linear block copolymers comprising a poly(alkylene glycol) block surrounded by two ethylenic blocks. These polymers have the drawback of having a central block of poly(alkylene glycol) type of high mass, which gives the polymer high crystallinity, which may lead to opaque products and/or products of greasy nature.

The Applicant has discovered novel polymers that can give a styling and conditioning effect to cosmetic haircare products.

Without wishing to be bound by the present explanation, it may be considered that this may be due especially to the presence of PEG (meth)acrylate (MPEG) units in the polymer chain, these units largely contributing to the obtained effect. Specifically, it has been found that this effect is not obtained with a simple mixture of cationic polymer and of polymer of PEG type.

Surprisingly, the polymers according to the invention have advantageous cosmetic properties, for example during application in a formulation of hair conditioner type in combination with conditioning agents; specifically, it has been found that the hair disentangles easily during rinsing, and is soft; after drying, the compositions according to the invention also allow, once the hair has dried, particularly advantageous shaping of the hair.

The cosmetic compositions according to the invention are characterized in that they comprise:
I) at least one conditioning agent chosen from synthetic oils, mineral oils, plant oils, fluoro or perfluoro oils, natural or synthetic waxes, silicones, cationic polymers with a cationic charge density of less than 5 meq/g, cationic proteins, cationic protein hydrolyzates, compounds of ceramide type, cationic surfactants, fatty amines, fatty acids and derivatives thereof, and also mixtures of these various compounds, and
II) at least one ethylenic copolymer comprising, as a weight percentage relative to the total weight of the polymer:
a) 10-60% by weight of one or more monomers of formula (I) as defined below;
b) 40-90% by weight of at least one “essentially cationic” monomer chosen from:
   (i) one or more cationic monomers of formula (IIa),
   (ii) one or more amphoteric monomers of formulae (IIc) and (IId), and
   (iii) a mixture of one or more cationic monomers of formula (IIa) with one or more anionic monomers chosen from maleic anhydride and/or those of formula (IIb); and/or with one or more amphoteric monomers chosen from those of formulae (IIc) and (IId),
c) and optionally 0-50% by weight of nonionic hydrophilic monomers, excluding methyl acrylate, methyl methacrylate and isopropyl acrylate if they are present in an amount of greater than or equal to 10% by weight.

In the rest of the present description, the term “cyclic radical” means a monocyclic or polycyclic radical, which may be in the form of one or more saturated and/or unsaturated, optionally substituted rings (for example cyclohexyl, cyclodecyl, benzyl or fluorenyl), but also a radical that comprises one or more of the said rings (for example p-tert-butyl-cyclohexyl or 4-hydroxybenzyl).

The term “saturated and/or unsaturated radical” means totally saturated radicals, totally unsaturated radicals, including aromatic radicals, and also radicals comprising one or more double and/or triple bonds, the rest of the bonds being single bonds.

The ethylenic copolymer according to the invention thus comprises at least one monomer of formula (I), which may be present alone or as a mixture:

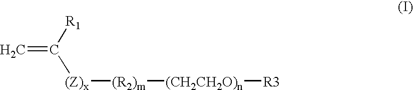

in which:
R1 is a hydrogen atom or a linear or branched hydrocarbon-based radical, of the type CₚH₂ₚ₊₁, with p being an integer between 1 and 12 inclusive;
Z is a divalent group chosen from —COO—, —CONH—, —CONCH₃—, —OCO—, —O—, —SO₂—, —CO—O—CO— and —CO—CH₂—CO—;
x is 0 or 1;
R2 is a saturated or unsaturated, optionally aromatic, linear, branched or cyclic carbon-based divalent radical of 1 to 30 carbon atoms, possibly comprising 1 to 18 heteroatoms chosen from O, N, S, F, Si and P;
m is 0 or 1;
n is an integer between 3 and 300 inclusive;
R3 is a hydrogen atom or a saturated or unsaturated, optionally aromatic, linear, branched or cyclic carbon-based radical of 1 to 30 carbon atoms, possibly comprising 1 to 20 heteroatoms chosen from O, N, S, F, Si and P;
   and salts thereof.

R1 may especially represent a methyl, ethyl, propyl or butyl radical. Preferably R1 represents hydrogen or a methyl radical.

Preferably, Z represents COO or CONH.

Preferably, x is equal to 1.

In the radical R2, the heteroatom(s), when they are present, may be intercalated in the chain of said radical R2, or alternatively said radical R2 may be substituted with one or more groups comprising them such as hydroxy or amino (NH2, NHR′ or NR′R″ with R′ and R″, which may be identical or different, representing a linear or branched C1-C22 alkyl, especially methyl or ethyl).

R2 may especially be:
an alkylene radical such as methylene, ethylene, propylene, n-butylene, isobutylene, tert-butylene, n-hexylene, n-octylene, n-dodecylene, n-octadecylene, n-tetradecylene or n-docosanylene;
a phenylene radical —C₆H₄— (ortho, meta or para), optionally substituted with a C1-C12 alkyl radical optionally comprising 1 to 25 heteroatoms chosen from O, N, S, F, Si and P; or alternatively a benzylene radical —C₆H₄—CH₂—, optionally substituted with a C1-C12 alkyl radical optionally comprising 1 to 8 heteroatoms chosen from O, N, S, F, Si and P;
a pyridinium radical of formula:

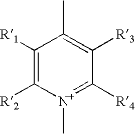

with R′1 to R′4, which may be identical or different, chosen from H and a C1-C12 alkyl radical optionally comprising 1 to 8 heteroatoms chosen from O, N, S, F, Si and P; R′1 to R′4 may especially be methyl and/or ethyl;
a radical of formula —CH₂—O—CO—O—, CH₂—CH₂—O—CO—O—, —CH₂—CO—O—, —CH₂—CH₂—CO—O—, —CH₂—O—CO—NH—, —CH₂—CH₂—O—CO—NH—; —CH₂—NH—CO—NH—, —CH₂—CH₂—NH—CO—NH—; —CH₂—CHOH—, —CH₂—CH₂—CHOH—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, —CH₂—CH₂—CH₂—NR′—, —CH₂—CH₂—CH₂—O—; —CH₂—CH₂—CHR′—O— with R′ and R″ representing a linear or branched C1-C22 alkyl optionally comprising 1 to 12 heteroatoms chosen from O, N, S, F, Si and P;
or a mixture of these radicals.

Preferably, n is between 5 and 200 inclusive and better still between 7 and 100 inclusive, or even between 9 and 50 inclusive.

Preferably, R3 is a hydrogen atom; a benzyl or phenyl radical optionally substituted with a C1-C12 alkyl radical optionally comprising 1 to 8 heteroatoms chosen from O, N, S, F, Si and P; a C1-C30 and especially C1-C22 or even C2-C16 alkyl radical, optionally comprising 1 to 18 heteroatoms chosen from O, N, S, F, Si and P.

These benzyl, phenyl or alkyl radicals may especially comprise a function chosen from the following functions:

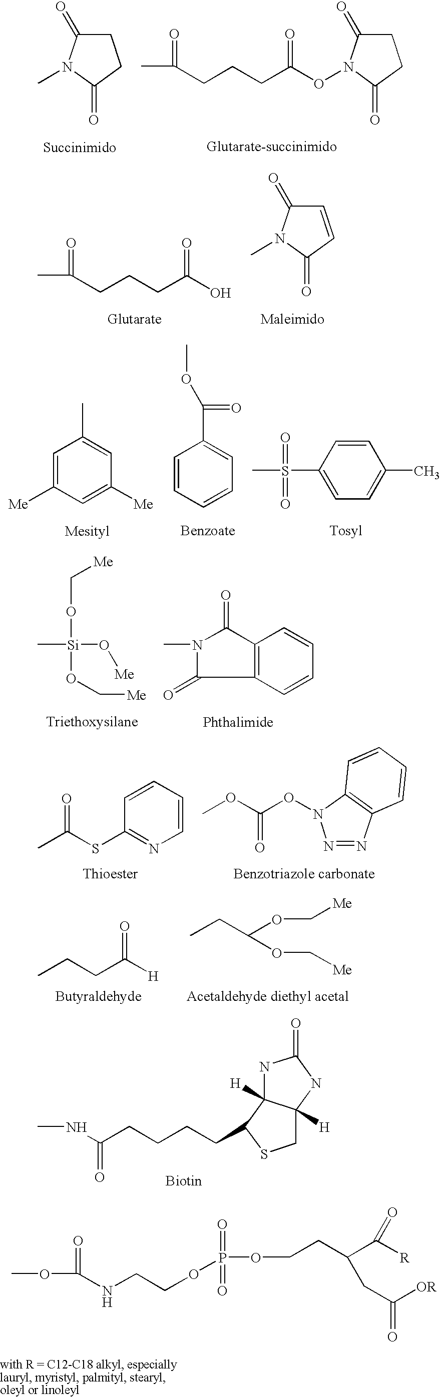

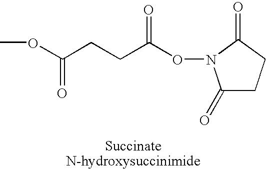

or alternatively chosen from —SO₃H, —COOH, —PO₄, —NR5R6 and —N⁺R5R6R7, with R5, R6 and R7, independently of each other, chosen from H and linear, branched or cyclic C1-C18 alkyls, especially methyl optionally comprising one or more heteroatoms or alternatively bearing protective groups such as t-butyloxycarbonyl (also known as BOC) or 9-fluorenylmethoxycarbonyl (also known as FmoC).

Among the radicals R3, mention may be made of methyl, ethyl, propyl, benzyl, ethylhexyl, lauryl, stearyl and behenyl (—(CH₂)₂₁—CH₃) chains, and also fluoroalkyl chains, for instance heptadecafluorooctylsulfonyl-aminoethyl CF₃—(CF₂)₇—SO₂—N(C₂H₅)—CH₂—CH₂; or alternatively —CH₂—CH₂—CN, succinimido, maleimido, mesityle, tosyl, triethoxysilane or phthalimide chains.

The amine units and/or the anionic groups of the monomer of formula (I) may optionally be neutralized.

The amine units of the monomer may optionally be neutralized.

Among the salts, mention may be made of the salts of mineral acids, such as sulfuric acid, hydrochloric acid, hydrobromic acid, hydroiodic acid, phosphoric acid or boric acid. Mention may also be made of the salts of organic acids, which may comprise one. Or more carboxylic, sulfonic or phosphonic acid groups. These may be linear, branched or cyclic aliphatic acids or alternatively aromatic acids. These acids may also comprise one or more heteroatoms chosen from O and N, for example in the form of hydroxyl groups. Mention may be made especially of propionic acid, acetic acid, terephthalic acid, citric acid and tartaric acid.

Neutralization of the anionic groups may be performed with a mineral base such as LiOH, NaOH, KOH, Ca(OH)₂, NH₄OH, Mg(OH)₂ or Zn(OH)₂; or with an organic base such as a primary, secondary or tertiary alkylamine, especially triethylamine or butylamine. This primary, secondary or tertiary alkylamine may comprise one or more nitrogen and/or oxygen atoms and may thus comprise, for example, one or more alcohol functions; mention may be made especially of 2-amino-2-methyl-propanol, triethanolamine and dimethylamino-2-propanol. Mention may also be made of lysine or 3-(dimethylamino)propylamine.

Among the monomers of formula (I) that are particularly preferred, mention may be made of:
poly(ethylene glycol) (meth)acrylate in which R1 is H or methyl; Z is COO, x=1, m=0 and R3=H;
methylpoly(ethylene glycol) (meth)acrylate, also known as methoxypoly(ethylene glycol) (meth)acrylate, in which R1 is H or methyl, Z is COO, x=1, m=0 and R3=methyl;
alkylpoly(ethylene glycol) (meth)acrylates in which R1 is H or methyl, Z is COO, x=1, m=0 and R3=alkyl;
phenylpoly(ethylene glycol) (meth)acrylate, also known as poly(ethylene glycol) phenyl ether (meth)acrylate, in which R1 is H or methyl, Z is COO, x=1, m=0 and R3=phenyl;
the following monomer:

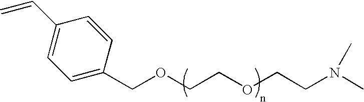

in which n is preferably between 3 and 100 inclusive and especially 5 to 50 inclusive, or even 7 to 30 inclusive.

The monomers of formula (I) that are most particularly preferred are chosen from poly(ethylene glycol) (meth)acrylates and methylpoly(ethylene glycol) (meth)acrylates, preferably those with a molecular weight of between 350 and 15 000 g/mol and especially between 500 and 8000 g/mol.

Poly(ethylene glycol) (meth)acrylates are most particularly preferred, and in particular those with a molecular weight of between 350 and 15 000 g/mol and especially between 500 and 8000 g/mol.

### Examples of Commercial Monomers are

CD 350 (methoxypoly(ethylene glycol 350) methacrylate) and CD 550 (methoxypoly(ethylene glycol 550) methacrylate), sold by Sartomer Chemicals;
M90G (methoxypolyethylene glycol methacrylate (9 repeater units)) and M230G (methoxypolyethylene glycol methacrylate (23 repeating units)) available from Shin-Nakamura Chemicals;
methoxypoly(ethylene glycol) methacrylates with average molecular weights of 300, 475 or 1100, available from Sigma-Aldrich;
methoxypoly(ethylene glycol)acrylate with an average molecular weight of 426, available from Sigma-Aldrich;
methoxypoly(ethylene glycol) methacrylates available from Laporte under the trade names: MPEG 350, MPEG 550, S10W, S20W;
poly(ethylene glycol) monomethylether, mono(succinimidyl succinate) ester with an average molecular weight of 1900 or 5000, from Polysciences;
behenylpoly(ethylene glycol PEG-25) methacrylate, available from Rhodia under the name Sipomer BEM;
poly(ethylene glycol)phenylether acrylates with average molecular weights of 236; 280 or 324, available from Aldrich;
methoxypolyethylene glycol 5000 2-(vinylsulfonyl)ethyl ether available commercially from Fluka;
polyethylene glycol ethyl ether methacrylate available from Aldrich;
polyethylene glycol 8000, 4000, 2000 methacrylates from Monomer & Polymer Dajac Laboratories;
polyethylene glycol N-hydroxysuccinimide vinyl sulfone available commercially from Nektar Molecule Engineering (Shearwater).

Preferably, the monomer of formula (I) has a molecular weight of between 350 and 15 000 g/mol and especially between 500 and 8000 g/mol.

The monomer of formula (I), alone or as a mixture, is present in a proportion of from 10% inclusive to 60% exclusive by weight, especially from 20% inclusive to 55% inclusive by weight and preferably from 30% inclusive to 50% inclusive by weight, relative to the weight of the final polymer.

### Ionic Units

The ethylenic copolymer according to the invention also comprises at least one “essentially cationic” monomer, or a salt thereof, chosen from:
(i) one or more cationic monomers of formula (IIa),
(ii) one or more amphoteric monomers of formulae (IIc) and (IId), and
(iii) a mixture of one or more cationic monomers of formula (IIa) with one or more anionic monomers chosen from maleic anhydride and/or those of formula (IIb); and/or with one or more amphoteric monomers chosen from those of formulae (IIc) and (IId).

Preferably, the “essentially cationic” monomer is chosen from the cationic monomers of formula (IIa) and the amphoteric monomers of formula (IIc) or (IId) and preferentially from the cationic monomers of formula (IIa).

The term “cationic monomer” means a monomer comprising units capable of bearing a cationic charge in the pH range of between 3 and 12. These units do not necessarily have a permanent charge irrespective of the pH. The cationic unit does not need to be protonated at each of these pH values.

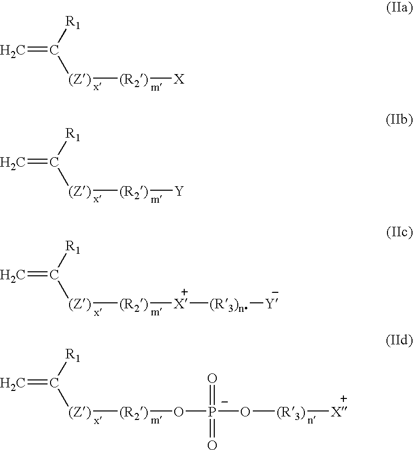

in which:
R1 is a hydrogen atom or a linear or branched hydrocarbon-based radical of the type CₚH₂ₚ₊₁, with p being an integer between 1 and 12 inclusive.

R1 may especially represent a methyl, ethyl, propyl, isopropyl, n-butyl, isobutyl or tert-butyl radical.

Preferably, R1 represents hydrogen or a methyl radical.
Z¹ is a divalent group chosen from —COO—, —CONH—, —CONCH₃—, —OCO— or —O—, —SO₂—CO—O—CO— or —CO—CH₂—CO—.

Preferably, Z′ is chosen from COO and CONH.
x′ is 0 or 1, preferably 1.
R′2 is a saturated or unsaturated, optionally aromatic, linear, branched or cyclic divalent carbon-based radical of 1 to 30 carbon atoms, possibly comprising 1 to 18 heteroatoms chosen from O, N, S, F, Si and P.

In the radical R′2 the heteroatom(s), when they are present, may be intercalated in the chain of said radical R′2, or alternatively said radical R′2 may be substituted with one or more groups comprising them such as hydroxyl or amino (NH2, NHR′ or NR′R″ with R″ and R″, which may be identical or different, representing a linear or branched C1-C22 alkyl, especially methyl or ethyl).

R′2 may especially be:
an alkylene radical such as methylene, ethylene, propylene, n-butylene, isobutylene, tert-butylene, n-hexylene, n-octylene, n-dodecylene, n-octadecylene, n-tetradecylene or n-docosanylene;
a phenylene radical —C₆H₄— (ortho, meta or para), optionally substituted with a C1-C12 alkyl radical optionally comprising 1 to 25 heteroatoms chosen from N, O, S, F, Si and/or P; or alternatively a benzylene radical —C₆H₄—CH₂—, optionally substituted with a C1-C12 alkyl radical optionally comprising 1 to 25 heteroatoms chosen from O, N, S, F, Si and P;
a radical of formula —CH₂—O—CO—O—, CH₂—CH₂—O—CO—O—, —CH₂—CO—O—, —CH₂—CH₂—CO—O—, —[(CH₂)₅—CO—O]ₙ, —CH₂—CH(CH₃)—O—, —(CH₂)₂—O—, —CH₂—O—CO—NH—, —CH₂—CH₂—O—CO—NH—; —CH₂—NH—CO—NH— or —CH₂—CH₂—NH—CO—NH—, —CH₂—CHOH—, —CH₂—CH₂—CHOH—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, —CH₂—CH₂—CH₂—NR′—, —C₂—CH₂—CH₂—O—; —CH₂—CH₂—CHR′—O— with R′ and R″ representing a linear or branched C1-C22 alkyl optionally comprising 1 to 12 heteroatoms chosen from O, N, S, F, Si and P;
or a mixture of these radicals;
m′ is 0 or 1;
X (in formula IIa) is a group of formula —N(R₆)(R₇) or —P(R₆)(R₇) or —P⁺R₆R₇R₈, with R6, R7 and R8 representing, independently of each other, either (i) a hydrogen atom, or (ii) a linear, branched or cyclic, saturated or unsaturated, optionally aromatic alkyl group containing from 1 to 18 carbon atoms, possibly comprising 1 to 10 heteroatoms chosen from O, N, S, F, Si and P; or (iii) R6 and R7 may form with the nitrogen or phosphorus atom a first saturated or unsaturated, optionally aromatic ring comprising in total 5, 6, 7 or 8 atoms, and especially 4, 5 or 6 carbon atoms and/or 2 to 4 heteroatoms chosen from O, S and N; said first ring possibly being fused with one or more other saturated or unsaturated, optionally aromatic rings each comprising 5, 6 or 7 atoms, and especially 4, 5, 6 or 7 carbon atoms and/or 2 to 4 heteroatoms chosen from O, S and N.

For example, R6 and R7 may be chosen from hydrogen and a methyl, ethyl, propyl, isopropyl, n-butyl, t-butyl, isobutyl, octyl, lauryl or stearyl group.

Preferably, R6 and R7 are chosen, independently of each other, from H, CH3 and C2H5.

Alternatively, X may represent a group —R′6-N—R′7- in which R′6 and R′7 form with the nitrogen atom a saturated or unsaturated, optionally aromatic ring comprising in total 5, 6, 7 or 8 atoms, and especially 4, 5 or 6 carbon atoms and/or 2 to 4 heteroatoms chosen from O, S and N; said ring possibly being fused with one or more other saturated or unsaturated, optionally aromatic rings, each comprising 5, 6 or 7 atoms, and especially 4, 5, 6, 7 or 8 carbon atoms and/or 2 to 4 heteroatoms chosen from O, S and N.

For example, X may constitute an aromatic or nonaromatic ring comprising a tertiary amine group or may represent an aromatic or nonaromatic heterocycle containing a tertiary nitrogen.

Among these preferred radicals X, mention may be made of radicals of pyridine, indolyl, isoindolinyl, imidazolyl, imidazolinyl, piperidyl, pyrazolinyl, pyrazolyl, quinoline, pyrazolinyl, pyridyl, piperazinyl, pyrrolidinyl, quinidinyl, thiazolinyl, morpholine, guanidino, amidino or phosphonium type, and mixtures thereof.

The guanidino and amidino groups are, respectively, of formula:

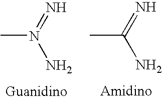

The monomers of formula (IIa) may be neutralized with neutralizers of different chemical nature.

The neutralizer may be chosen from mineral or organic acids in the Brönsted sense and preferably from organic acids. Advantageously, it may be chosen from neutralizers with a log P value of less than or equal to 2, for example between −8 and 2, preferably between −6 and 1 and especially between −6 and 0.

It may also be chosen from agents with a log P value of greater than 2, preferably greater than or equal to 2.5, especially greater than 3, and in particular between 3 and 15, or even between 3.5 and 10.

As stated hereinbelow, the log P values are known and are determined according to a standard test that determines the concentration of the neutralizer in 1-octanol and water.

The mineral acids that may be used are especially sulfuric acid, hydrochloric acid, hydrobromic acid, hydroiodic acid, phosphoric acid and boric acid.

The neutralizers of organic acid type may be chosen from linear, branched or cyclic aliphatic acids and/or unsaturated or aromatic acids, and may especially contain 1 to 1000 carbon atoms and especially 2 to 500 carbon atoms. They contain at least one acid function in the Brönsted sense, and especially one or more carboxylic, sulfonic and/or phosphonic acid groups. They may also comprise one or more heteroatoms chosen from O, N, Si, F and P, for example in the form of hydroxyl groups.

Neutralizers that may be used in particular include linear, branched or cyclic saturated or unsaturated, optionally aromatic fatty acids containing 6 to 32 and especially 8 to 28 carbon atoms, and comprising at least one COOH or sulfonic acid (—SO₃H) function.

Linear, branched or cyclic, saturated or unsaturated, optionally aromatic hydroxy acids, especially α-hydroxy acids, containing 6 to 32 and especially 8 to 28 carbon atoms, and comprising at least one COOH or sulfonic acid (—SO₃H) function may also be used.

Alkylbenzenesulfonic acids in which the alkyl group may contain from 4 to 30 and especially from 6 to 24 carbon atoms may also be used.

Amphoteric neutralizers, especially of the alkylbetaine or alkylamidopropylbetaine type, in which the alkyl group may contain 4 to 30 and especially 6 to 24 carbon atoms, may also be used; mention may be made in particular of cocoamidopropylbetaine.

Mention may be made especially of α-hydroxyethanoic acid, α-hydroxyoctanoic acid, α-hydroxycaprylic acid, ascorbic acid, acetic acid, benzoic acid, behenic acid, capric acid, citric acid, caproic acid, caprylic acid, dodecylbenzenesulfonic acid, 2-ethylcaproic acid, folic acid, fumaric acid, galactaric acid, gluconic acid, glycolic acid, 2-hexadecyleicosanoic acid, hydroxycaproic acid, 12-hydroxystearic acid, isolauric acid (or 2-butyloctanoic acid), isomyristic acid (or 2-hexyloctanoic acid), isoarachidic acid (or 2-octyldodecanoic acid), isolignoceric acid (or 2-decyltetradecanoic acid), lactic acid, lauric acid, malic acid, myristic acid, oleic acid, palmitic acid, propionic acid, sebacic acid, stearic acid, tartaric acid, terephthalic acid, trimesic acid, undecylenic acid, propylbetaine, cocoamidopropylbetaine, and betaine hydrochloride of formula [(CH₃)₃N+CH₂CO₂H.Cl—], and mixtures thereof.

Caproic acid, 2-ethylcaproic acid, oleic acid, behenic acid, stearic acid, acetic acid, citric acid, tartaric acid, betaine hydrochloride and/or gluconic acid is preferably used as neutralizer, and preferentially betaine hydrochloride and/or behenic acid.

The log P value of certain common acids is given below for information purposes:

| | |
|---|---|
| Sulfuric acid | −1.031 +/− 0.613 |
| Acetic acid | −0.285 +/− 0.184 |
| Propionic acid | 0.246 +/− 0.184 |
| Citric acid | −1.721 +/− 0.396 |
| Gluconic acid | −3.175 +/− 0.852 |
| Boric acid | −0.292 +/− 0.753 |
| Phosphoric acid | −2.148 +/− 0.587 |
| Benzoic acid | 1.895 +/− 0.206 |
| Stearic acid | 8.216 +/− 0.186 |
| Behenic acid | 10.342 +/− 0.186 |
| Oleic acid | 7.698 +/− 0.199 |

The term “neutralization” means the action of an organic acid according to the invention, and comprising at least one acid function in the Brönsted sense, on all or part of the monomers and/or polymer mentioned above, comprising at least one basic function in the Brönsted sense.

The neutralizer, alone or as a mixture, may be added in an amount of from 0.01 to 3 and especially 0.05 to 2.5 molar equivalents, or even 0.1 to 2 molar equivalents, relative to the total amine functions of the polymer or of the monomers.

It is thus possible to underneutralize the polymer, i.e. the neutralizer may be present in an amount necessary to neutralize 1% to 99%, especially 5% to 90% or even 10% to 80% of the total amine functions of the polymer or of the monomers; this means that it is present in an amount of from 0.01 to 0.99 and especially 0.05 to 0.9 molar equivalent, or even 0.1 to 0.8 molar equivalent.

It is also possible to overneutralize the polymer, i.e. the neutralizer may be present in an amount necessary to neutralize 101% to 300%, especially from 120% to 250% or even from 150% to 200% of the total amine functions of the polymer or of the monomers; this may be the case when it is desired to ensure that the polymer has a suitable pH range and/or ionic strength with respect to the envisioned formulations. It may thus be present in an amount of from 1.01 to 3 and especially 1.2 to 2.5 molar equivalents, or even 1.5 to 2 molar equivalents, relative to the total amine functions of the polymer or of the monomers.

Preferably, the neutralizer, alone or as a mixture, is present in a stoichiometric amount relative to the total amine functions of the polymer or of the monomers; it is thus present in an amount necessary to neutralize 100% of the amine units of the polymer or of the monomers, i.e. 1 molar equivalent.

Preferentially, the nature and amount of neutralizer may be determined by a person skilled in the art so as finally to obtain a water-soluble or water-dispersible polymer.

Among the preferred monomers of formula (IIa) that may be mentioned are:

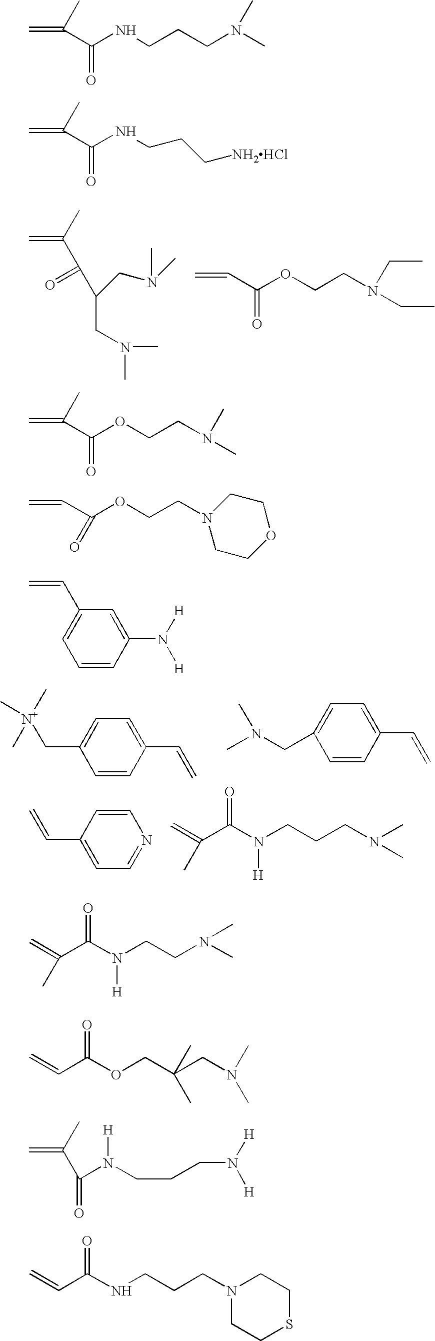

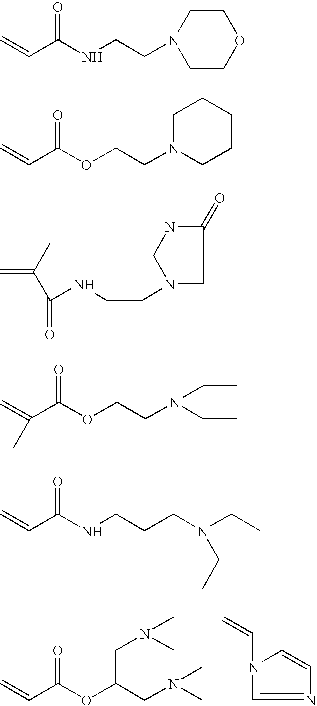

Among the monomers of formula (IIa) that are particularly preferred, mention may be made of dimethylaminopropyl(meth)acrylamide, dimethylaminoethyl(meth)acrylamide, diethylaminoethyl (meth)acrylate, dimethylaminoethyl(meth)acrylate, vinylimidazol, vinylpyridine and morpholinoethyl (meth)acrylate, and more particularly dimethylaminopropyl(meth)acrylamide.

In formula (IIb), the meaning of the radicals R1, Z′, x′, R′2 and m′ is the same as that given above for formula (IIa).

In formula (IIb), Y is a group chosen from —COOH, —SO₃H, —OSO₃H, —PO₃H₂ and —OPO₃H₂.

Neutralization of the anionic groups may be performed with a mineral base, such as LiOH, NaOH, KOH, Ca(OH)₂, NH₄OH, Mg(OH)₂ or Zn(OH)₂; or with an organic base such as a primary, secondary or tertiary alkylamine, especially triethylamine or butylamine. This primary, secondary or tertiary alkylamine may comprise one or more nitrogen and/or oxygen atoms and may thus comprise, for example, one or more alcohol functions; mention may be made especially of 2-amino-2-methylpropanol, triethanolamine and dimethylamino-2-propanol. Mention may also be made of lysine or 3-(dimethylamino)propylamine.

It is understood that, according to the prior art, the SO₄H₂ and PO₄H₂ groups are linked to R′2 via the oxygen atom, whereas the SO₃H and PO₃H groups are linked to R′2 via, respectively, the S and P atoms.

Among the anionic monomers that are preferred, mention may be made of maleic anhydride and the following preferred monomers of formula (IIb): acrylic acid, methacrylic acid, crotonic acid, itaconic acid, fumaric acid, maleic acid, 2-carboxyethyl acrylate (CH2=CH—C(O)—O—(CH₂)₂—COOH); styrenesulfonic acid, 2-acrylamido-2-methylpropanesulfonic acid, vinylbenzoic acid, vinylphosphoric acid, sulfopropyl(meth)acrylate, and the salts thereof.

In formula (IIc), the meaning of the radicals R1, Z′, x′, R′2 and m′ is the same as that given above for formula (IIa).

The other radicals have the following meaning:
X′⁺ is a divalent group of formula —N⁺(R₆)(R₇)— with R6 and R7 representing, independently of each other, either (i) a hydrogen atom, or (ii) a linear, branched or cyclic, optionally aromatic alkyl group containing from 1 to 25 carbon atoms, possibly comprising 1 to 20 heteroatoms chosen from O, N, S and P; or (iii) R6 and R7 may form with the nitrogen atom a first saturated or unsaturated, optionally aromatic ring comprising in total 5, 6, 7 or 8 atoms, and especially 4, 5, 6 or 7 carbon atoms and/or 2 to 3 heteroatoms chosen from O, S and N; said first ring possibly being fused with one or more other saturated or unsaturated, optionally aromatic rings, each comprising 5, 6, 7 or 8 atoms, and especially 4, 5, 6 or 7 carbon atoms and/or 2 to 3 heteroatoms chosen from O, S and N.

For example, R6 and R7 may be chosen from hydrogen and a methyl, ethyl, propyl, isopropyl, n-butyl, t-butyl or isobutyl group.

Among the preferred radicals X′⁺ that may be mentioned are radicals of pyridine indolyl, isoindolinyl, imidazolyl, imidazolinyl, piperidyl, pyrazolinyl, pyrazolyl, quinoline, pyrazolinyl, pyridyl, piperazinyl, pyrrolidinyl, quinidinyl, thiazolinyl, morpholine, guanidino or amidino type, and mixtures thereof.
Y′⁻ is a group chosen from —COO⁻, —SO₃⁻, —OSO₃⁻, PO₃²⁻ and —OPO₃²⁻.
R′3 is a saturated or unsaturated, optionally aromatic, linear, branched or cyclic divalent carbon-based radical of 1 to 30 carbon atoms, possibly comprising 1 to 18 heteroatoms chosen from O, N, S, F, Si and P.

In the radical R′3, the heteroatom(s), when they are present, may be intercalated in the chain of said radical R′3, or alternatively said radical R′3 may be substituted with one or more groups comprising them such as hydroxyl or amino (NH₂, NHR′ or NR′R″ with R′ and R″, which may be identical or different, representing a linear or branched C1-C18 alkyl, especially methyl or ethyl).

R′3 may especially be:
an alkylene radical such as methylene, ethylene, propylene, n-butylene, isobutylene, tert-butylene, n-hexylene, n-octylene, n-dodecylene, n-octadecylene, n-tetradecylene or n-docosanylene;
a phenylene radical —C₆H₄— (ortho, meta or para), optionally substituted with a C1-C12 alkyl radical optionally comprising 1 to 5 heteroatoms chosen from O, N, S, F, Si and P; or alternatively a benzylene radical —C₆H₄—CH₂—, optionally substituted with a C1-C12 alkyl radical optionally comprising 1 to 5 heteroatoms chosen from O, N, S, F, Si and P;
a radical of formula —CH₂—O—CO—O—, CH₂—CH₂—O—CO—O—, —CH₂—CO—O—, —CH₂—CH₂—CO—O—, —[(CH₂)₅—CO—O]ₙ—, —CH₂—CH(CH₃)—O—, —(CH₂)₂—O—, —CH₂—O—CO—NH—, —CH₂—CH₂—O—CO—NH—; —CH₂—NH—CO—NH— or —CH₂—CH₂—NH—CO—NH—, —CH₂—CHOH—, —CH₂—CH₂—CHOH—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, —CH₂—CH₂—CH₂—NR′—, —CH₂—CH₂—CH₂—O—; —[CH₂—CH₂—O]ₙ— and —[CH₂—CH(CH₃)—O]ₙ—, —CH₂—CH₂—CHR′—O— with R′ and R″ representing a linear or branched C1-C22 alkyl optionally comprising 1 to 12 heteroatoms chosen from O, N, S, F, Si and P;
or a mixture of these radicals;
n′ is between 1 and 100 and preferably between 1 and 5 inclusive.

In formula (IId), the meaning of the radicals R1, Z′, x′, R′2 and m′ is the same as that given above for formula (IIa), and that of the radicals R′3 and n′ is the same as that given for formula (IIc).

In formula (IId), X″⁺ is a group of formula —N⁺R₆R₇R₈ with R6, R7 and R8 representing, independently of each other, either (i) a hydrogen atom, or (ii) a linear, branched or cyclic, optionally aromatic alkyl group containing from 1 to 18 carbon atoms, possibly comprising 1 to 5 heteroatoms chosen from O, N, S and P; or (iii) R6 and R7 may form with the nitrogen atom a first saturated or unsaturated, optionally aromatic ring comprising in total 5, 6 or 7 atoms, and especially 4, 5 or 6 carbon atoms and/or 2 to 3 heteroatoms chosen from O, S and N; said first ring possibly being fused with one or more other saturated or unsaturated, optionally aromatic rings, each comprising 5, 6 or 7 atoms, and especially 4, 5, 6 or 7 carbon atoms and/or 2 to 3 heteroatoms chosen from O, S and N.

For example, R6, R7 and R8 may be chosen from hydrogen and a methyl, ethyl, propyl, isopropyl, n-butyl, t-butyl, isobutyl, octyl, lauryl or stearyl group.

Among the preferred radicals X″⁺ that may be mentioned are trimethylammonium; triethylammonium; N,N-dimethyl-N-octylammonium; N,N-dimethyl-N-laurylammonium radicals.

Among the preferred monomers of formula (IIc) or (IId) that may be mentioned are N,N-dimethyl-N-(2-methacryloyloxyethyl)-N-(3-sulfopropyl)ammonium betaine (especially SPE from the company Raschig); N,N-dimethyl-N-(3-methacrylamidopropyl)-N-(3-sulfopropyl)ammonium betaine (SPP from Raschig) and 1-(3-sulfopropyl)-2-vinylpyridinium betaine (SPV from Raschig), and also 2-methacryloyloxyethylphosphorylcholine.

When the “essentially cationic” monomer is chosen from mixtures of cationic and/or amphoteric monomers with anionic monomers, said anionic monomers are preferably present in a proportion of from 5% to 40% by weight, especially from 10% to 30% by weight and preferably from 15% to 25% by weight relative to the weight of the “cationic and/or amphoteric+anionic” mixture.

The “essentially cationic” monomer is present in a proportion of from 40% to 90% by weight, especially from 45% to 80% by weight and preferably from 50% to 70% by weight relative to the weight of the final polymer.

Preferably, the polymer according to the invention comprises the monomers of formula (I) and the ionic monomers (the cationic monomers+the optional amphoteric and anionic monomers) in a weight ratio that may range from 60/40 to 40/60, with a preference for a 50/50 ratio.

The ethylenic copolymer according to the invention may optionally comprise monomers other than those mentioned above. These additional monomers are thus nonionic.

When it comprises such additional monomers, they are necessarily chosen from the monomers that are “hydrophilic” within the meaning of the present invention.

The term “hydrophilic monomer” means monomers with a value of the logarithm of the 1-octanol/water apparent partition coefficient, also known as the log P, of less than or equal to 2, for example between −8 and 2, preferably less than or equal to 1.5, especially less than or equal to 1 and in particular between −7 and 1, or even between −6 and 0.

The log P values are known and determined according to a standard test that determines the concentration of the monomer in 1-octanol and water.

The values may especially be calculated using the ACD software (Advanced Chemistry Development) software solaris V4.67; they may also be obtained from Exploring QSAR: hydrophobic, electronic and stearic constants (ACS professional reference book, 1995).

A website also exists that provides estimated values (address: http://esc.syrres.com/interkow/kowdemo.htm).

The log P value of certain common monomers, determined using the ACD software, are given below:

| | Methacrylate | Acrylate |
|---|---|---|
| | (* or methacrylamide) | (* or acrylamide) |
| Methyl (meth)acrylate | 1.346 +/− 0.250 | 0.793 +/− 0.223 |
| Ethyl (meth)acrylate | 1.877 +/− 0.250 | 1.325 +/− 0.223 |
| Propyl (meth)acrylate | 2.408 +/− 0.250 | 1.856 +/− 0.223 |
| Isopropyl (meth)acrylate | 2.224 +/− 0.254 | 1.672 +/− 0.228 |
| Butyl (meth)acrylate | 2.940 +/− 0.250 | 2.387 +/− 0.223 |
| Isobutyl (meth)acrylate | 2.756 +/− 0.254 | 2.208 +/− 0.228 |
| Tert-butyl (meth)acrylate | 2.574 +/− 0.261 | 2.022 +/− 0.238 |
| Cyclohexyl (meth)acrylate | 3.405 +/− 0.252 | 2.853 +/− 0.226 |
| Octyl (meth)acrylate | 5.065 +/− 0.521 | 4.513 +/− 0.224 |
| Lauryl (meth)acrylate | 7.190 +/− 0.251 | 6.638 +/− 0.224 |
| Tridecyl (meth)acrylate | 7.712 +/− 0.251 | 7.170 +/− 0.224 |
| Cetyl (meth)acrylate | 9.316 +/− 0.251 | 8.764 +/− 0.224 |
| Palmityl (meth)acrylate | >9 | >9 |
| Stearyl (meth)acrylate | 10.379 +/− 0.251 | 9.826 +/− 0.224 |
| Behenyl (meth)acrylate | 11.952 +/− 0.225 | 12.504 ± 0.251 |
| Oleyl (meth)acrylate | >9 | 9.308 ± 0.232 |
| Tetrahydrofurfuryl | 1.352 ± 0.283 | 0.800 ± 0.263 |
| (meth)acrylate | | |
| 2-Ethyl hexyl | 4.881 ± 0.254 | 4.329 ± 0.229 |
| (meth)acrylate | | |
| 2-Hydroxyethyl | 0.718 ± 0.277 | 0.166 ± 0.258 |
| (meth)acrylate | | |
| Ethoxyethyl | 1.887 ± 0.293 | 1.335 ± 0.268 |
| (meth)acrylate | | |
| Hydroxypropyl | | 0.383 ± 0.241 |
| (meth)acrylate | | |
| N-isopropyl | 0.748 ± 0.276 | 0.195 ± 0.256 |
| (meth)acrylamide* | | |
| N-octyl (meth)acrylamide* | 3.558 ± 0.273 | 3.036 ± 0.253 |
| N,N-dimethyl | 0.906 ± 0.553 | −0.168 ± 0.556 |
| (meth)acrylamide* | | |
| N,N-dibutyl | 3.573 ± 0.570 | 3.021 ± 0.557 |
| (meth)acrylamide* | | |

| | |
|---|---|
| Vinyl acetate | 0.730 ± 0.286 |
| Methyl vinyl ether | 0.509 ± 0.286 |
| Ethyl vinyl ether | 1.040 ± 0.286 |
| Vinylcaprolactam | 1.499 ± 0.207 |
| Vinylpyrrolidone | 0.370 ± 0.206 |
| N-vinylacetamide | 0 ± 0.231 |

The additional hydrophilic monomers may be chosen especially from those of formula (III), alone or as a mixture:

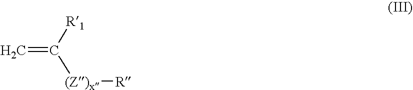

in which:
R′₁ is hydrogen or —CH₃;
Z″ is a divalent group chosen from —COO—, —CONH—, —CONCH₃—, —OCO—, —SO₂, —CO—O—CO—, —CO—CH₂—CO— and —O—; preferably COO and CONH;
x″ is 0 or 1;
R″ is a saturated or unsaturated, optionally aromatic, linear, branched or cyclic carbon-based radical of 1 to 30 carbon atoms, possibly comprising 1 to 18 heteroatoms chosen from O, N, S, F, Si and P.

In the radical R″, the heteroatom(s), when they are present, may be intercalated in the chain of said radical, or alternatively said radical may be substituted with one or more groups comprising them, such as hydroxyl, ester, amide, urethane or urea.

R″ may especially be a methyl, ethyl, propyl, isopropyl, n-butyl, isobutyl, tert-butyl, phenyl or benzyl radical, or a radical of formula —CH₂—CH₂—CH₂OH, —CH₂—CH₂—OH, —CH₂—CH₂—CH₂OH or furfuryl.

The additional hydrophilic nonionic monomers are especially chosen from the following monomers: methyl methacrylate, methyl acrylate, ethyl methacrylate, ethyl acrylate, propyl acrylate, isopropyl acrylate, tetrahydrofurfuryl methacrylate, tetrahydrofurfuryl acrylate, 2-hydroxyethyl methacrylate, 2-hydroxyethyl acrylate, ethoxyethyl methacrylate, ethoxyethylacrylate, N-isopropylacrylamide, N-isopropylmethacrylamide, N,N-dimethylacrylamide, N,N-dimethylmethacrylamide, vinyl acetate, methyl vinyl ether, ethyl vinyl ether, vinylpyrrolidone, vinylcaprolactam, N-vinylacetamide, hydroxylpropyl acrylate, N-vinyllactam, acrylamide, N-methylacrylamide, N,N-dimethylacrylamide, N-methyl-N-vinylacetamide, N-vinylformamide, N-methyl-N-vinylformamide, vinyl alcohol (copolymerized in the form of vinylacetate and then hydrolyzed).

The additional monomer, alone or as a mixture, may not be present in the polymer according to the invention (0%), or alternatively may be present in an amount that may range up to 50% by weight, relative to the weight of the final polymer; it may especially be present in an amount of from 0.1% to 35% by weight, preferably from 1% to 25% by weight, for example from 3% to 15% by weight, or even from 5% to 9.5% by weight, relative to the total weight of the polymer.

However, it has been found that when this additional monomer is chosen from methyl acrylate, methyl methacrylate and isopropyl acrylate, these monomers could not be present in an amount of greater than or equal to 10% by weight. These monomers may thus be present in a proportion of 0-9.5% by weight, especially from 0.1% to 8% by weight and preferably from 1% to 5% by weight in the final polymer.

Preferably, the copolymer according to the invention does not comprise any monomers other than those of formulae (I), (IIa), (IIb), (IIc) and (IId). Preferably, the copolymer according to the invention comprises only monomers of formulae (I) and (IIa).

Preferably, the polymer that may be used according to the invention comprises the monomers of formula (I) and the “essentially cationic” monomers in a weight ratio that may range from 60/40 to 40/60, with a preference for a 50/50 ratio.

In one particular embodiment of the invention, the polymer consists essentially of monomer of formula (I), alone or as a mixture, and of monomers of formula (IIa), alone or as a mixture.

The polymers that are most particularly preferred are those in which:
the monomer of formula (I), alone or as a mixture, is present in a proportion of from 10% to 60% by weight, especially from 20% to 60% by weight and preferably from 30%, to 50% by weight, relative to the weight of the final polymer, and is chosen, alone or as a mixture, from poly(ethylene glycol) (meth)acrylates, preferably those with a molecular weight of between 350 and 13 000 g/mol and especially between 500 and 8000 g/mol; and
the “essentially cationic” monomer is present in a proportion of from 40% to 90% by weight, especially from 40% to 80% by weight and preferably from 50% to 70% by weight relative to the weight of the final polymer, and is chosen, alone or as a mixture, from dimethylaminopropyl(meth)acrylamide, dimethylaminoethyl (meth)acrylamide, diethylaminoethyl (meth)acrylate, dimethylaminoethyl(meth)acrylate, vinylimidazole, vinylpyridine and morpholinoethyl (meth)acrylate; and
the polymer preferably being neutralized with an organic acid neutralizer chosen especially from 2-ethylcaproic acid, oleic acid, behenic acid, stearic acid, acetic acid, citric acid, tartaric acid, betaine hydrochloride and/or gluconic acid, and preferentially behenic acid and/or betaine hydrochloride.

The polymers that are even more particularly preferred are those in which:
the monomer of formula (I), alone or as a mixture, is present in a proportion of from 10% to 60% by weight, especially from 20% to 60% by weight and preferably from 30% to 50% by weight, relative to the weight of the final polymer, and is chosen, alone or as a mixture, from poly(ethylene glycol) (meth)acrylates, preferably those with a molecular weight of between 350 and 13 000 g/mol and especially between 500 and 8000 g/mol; and
the “essentially cationic” monomer is present in a proportion of from 40% to 90% by weight, especially from 40% to 80% by weight and preferably from 50% to 70% by weight, relative to the weight of the final polymer, and is chosen, alone or as a mixture, from dimethylaminopropyl(meth)acrylamide, and
the polymer being neutralized with a neutralizer chosen from behenic acid and/or betaine hydrochloride.

The polymers according to the invention may be prepared according to the usual standard radical polymerization methods known to those skilled in the art, and as described, for example, in the book “Chimie et physicochimie des polymères” by Gnanou et al. (published by Dunod).

These polymers may especially be prepared by:
direct solution polymerization in water with optional preneutralization of the cationic unit and/or of the anionic unit;
emulsion polymerization in water with optional preneutralization of the cationic unit and/or of the anionic unit, with use of a surfactant;
polymerization in an organic solvent, such as ethanol or methyl ethyl ketone, with optional preneutralization of the cationic unit and/or of the anionic unit, followed by a step of dissolution or dispersion in water with evaporation of the solvent.

These polymerizations may be performed in the presence of a radical initiator especially of peroxide type (Trigonox 21S: tert-butyl peroxy-2-ethylhexanoate) or azo type (AIBN V50: 2,2′-azobis(2-amidinopropane) dihydrochloride), which may be present in a proportion of from 0.3% to 5% by weight relative to the total weight of the monomers.

The polymers according to the invention are noncrosslinked. They are in the form of statistical, preferably film-forming, ethylenic copolymers of one more ethylenic monomers containing PEG groups (the PEG groups are pendent along the backbone) and of one or more ethylenic monomers comprising cationic functions (nonquaternary neutralized amines) and/or betaine functions and, optionally, one or more other monovalent nonionic hydrophilic ethylenic comonomers.

The term “ethylenic” polymer means a polymer obtained by polymerization of ethylenically unsaturated monomers.

The term “film-forming” polymer means a polymer that can form, by itself or in the presence of an auxiliary film-forming agent, a continuous film that adheres to a support, especially to keratin materials.

They have a weight-average molecular mass (Mw) that is preferably between 500 and 5 000 000, especially between 1000 and 3 000 000 and more preferentially between 2000 and 2 000 000, or even between 4000 and 500 000, better still between 7000 and 250 000 and even better between 8000 and 100 000.

The weight-average molar masses (Mw) are determined by gel permeation chromatography or by light scattering, depending on the accessibility of the method (solubility of the polymers under consideration).

The polymers that may be used according to the invention may preferably be conveyed in aqueous medium, i.e. they are preferably water-soluble or water-dispersible.

The term “water-soluble” means that it is soluble in water, to a proportion of at least 5% by weight, at 25° C., and forms a clear solution.

The term “water-dispersible” means that it forms in water, at a concentration of 5% by weight, at 25° C., a stable dispersion of fine, generally spherical particles. The mean size of the particles constituting said dispersion is less than 1 μm and more generally ranges between 5 and 400 nm and preferably from 10 to 250 nm. These particle sizes are measured by light scattering.

The dissolution or dispersion in water may be performed by direct dissolution of the polymer if it is soluble, or alternatively by neutralization of the amine and/or acid units so as to make the polymer soluble or dispersible in water. The dissolution or dispersion in water may also be performed via an intermediate step of dissolution in an organic solvent followed by the addition of water before evaporation of the organic solvent.

Moreover, it has been found that the polymers that may be used according to the invention advantageously have a viscosity in water that is adequate for the envisioned applications, which may be, for example, between 1 and 1000 mPa·s, preferably between 1.5 and 750 mPa·s and better still between 2 and 500 mPa·s.

The viscosity is measured using a Brookfield viscometer, for a solution containing 15% by weight of polymer in water or methyl ethyl ketone (solvent chosen as a function of the solubility of the polymer and/or of the polymerization method), at 25° C., with a needle-type spindle chosen from the model numbers 00 to 07 from Brookfield, preferably a No. 1 spindle; for a measuring time of 5 minutes, at a speed of between 0.1 and 6 rpm. The viscosity is measured after total dissolution of the polymer in water or methyl ethyl ketone.

In addition, the polymers that may be used according to the invention may preferably have a glass transition temperature (Tg) of between −150° C. and 20° C., especially −120° C. and 10° C. and better still between −100° C. and 0° C.; the Tg is measured according to the method given before the examples.

The polymers that may be used according to the invention may preferably have a melting point (m.p.) of between −100° C. and 80° C., especially between −80° C. and 50° C. and better still between −70° C. and 45° C., or even between −10° C. and 25° C.

In addition, the polymers that may be used according to the invention preferably have a water uptake of between 3% and 150% by weight, preferably between 4% and 100% by weight and especially between 5% and 50% by weight, at 75% relative humidity (75% HR); the water uptake is measured according to the method given before the examples.

They may also have a water uptake of between 3% and 20% by weight, preferably between 2.5% and 150% by weight and especially between 3% and 100% by weight, at 85% relative humidity (85% HR).

The polymers may be present in the composition in dissolved form, for example in water or an organic solvent, or alternatively in the form of an aqueous or organic dispersion.

They may be used in the cosmetic compositions according to the invention in a proportion of from 0.01% to 30% by weight of solids, especially from 0.1% to 20% by weight or even from 0.1% to 10% by weight and better still from 0.5% to 3% by weight relative to the total weight of the composition.

In the context of the present patent application, the term “conditioning agent” means any agent whose function is to improve the cosmetic properties of the hair, in particular the softness, sheen, disentangling, feel, smoothness and static electricity.

The conditioning agents may be in liquid, semisolid or solid form such as, for example, oils, waxes or gums.

According to the invention, the conditioning agents may be chosen from synthetic oils such as polyolefins, mineral oils, plant oils, fluoro oils or perfluoro oils, natural or synthetic waxes, silicones, cationic polymers, cationic proteins, cationic protein hydrolyzates, compounds of ceramide type, cationic surfactants, fatty amines, fatty acids and derivatives thereof, and also mixtures of these various compounds.

The conditioning agents that are preferred according to the invention are cationic polymers, cationic surfactants and silicones, and mixtures of these compounds.

The synthetic oils are especially polyolefins, in particular poly-α-olefins and more particularly:
of hydrogenated or nonhydrogenated polybutene type, and preferably hydrogenated or nonhydrogenated polyisobutene type.

Isobutylene oligomers with a molecular weight of less than 1000 and mixtures thereof with polyisobutylenes with a molecular weight of greater than 1000, and preferably between 1000 and 15 000, are preferably used.

As examples of poly-α-olefins that can be used in the context of the present invention, mention may be made more particularly of the polyisobutenes sold under the name Permethyl 99 A, 101 A, 102 A, 104 A (n=16) and 106 A (n=38) by the company Presperse Inc., or alternatively the products sold under the name Arlamol HD (n=3) by the company ICI (n denoting the degree of polymerization),
of hydrogenated or nonhydrogenated polydecene type.

Such products are sold, for example, under the names Ethylflo by the company Ethyl Corp. and Arlamol PAO by the company ICI.

The mineral oils that may be used in the compositions of the invention are preferentially chosen from the group formed by:
hydrocarbons, such as hexadecane and liquid paraffin.

The animal or plant oils are especially chosen from C₈-C₃₀ fatty acid triglycerides and preferentially from sweet almond oil, avocado oil, castor oil, olive oil, jojoba oil, sunflower oil, wheatgerm oil, sesameseed oil, groundnut oil, grapeseed oil, soybean oil, sesameseed oil, rapeseed oil, safflower oil, coconut oil, corn oil, hazelnut oil, shea butter, almond oil, apricot kernel oil, beauty-leaf oil, fish oils, glyceryl tricaprocaprylate, or plant or animal oils of formula R₉COOR₁₀ in which R₉ represents an acyl radical containing from 8 to 30 carbon atoms and R₁₀ represents a linear or branched hydrocarbon-based chain containing from 3 to 30 carbon atoms, in particular alkyl or alkenyl, for example purcellin oil or liquid jojoba wax.

It is also possible to use natural or synthetic essential oils such as, for example, eucalyptus oil, lavandin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil.

The waxes are natural (animal or plant) or synthetic substances that are solid at room temperature (20°-25° C.). They are insoluble in water, soluble in oils and are capable of forming a water-repellent film.

For the definition of waxes, mention may be made, for example, of P. D. Dorgan, Drug and Cosmetic Industry, December 1983, pp. 30-33.

The wax(es) is (are) chosen in particular from carnauba wax, candelilla wax, alfalfa wax, paraffin wax, ozokerite, plant waxes such as olive tree wax, rice wax, hydrogenated jojoba wax or the absolute waxes of flowers such as the essential wax of blackcurrant flower sold by the company Bertin (France), animal waxes such as beeswaxes, or modified beeswaxes (cerabellina); other waxes or waxy starting materials which can be used according to the invention are, in particular, marine waxes such as the product sold by the company Sophim under the reference M82, and polyethylene waxes or polyolefin waxes in general.

The cationic polymers that may be used according to the invention have a cationic charge density of less than 5 meq/g, preferably ranging, from 0.1 to 4 meq/g, more particularly from 0.2 to 3 meq/g and even more particularly from 0.3 to 2 meq/g. The charge density may be determined according to the Kjeldahl method. It is generally measured at a pH of the order of 3 to 9.

The cationic polymers, other than the polymers with a PEG group, that may be used in accordance with the present invention may be chosen from any of those already known per se as improving the cosmetic properties of the hair, i.e. especially those described in patent application EP-A-0 337 354 and in French patent applications FR-A-2 270 846, 2 383 660, 2 598 611, 2 470 596 and 2 519 863 and having a cationic charge density as defined above.

Even more generally, for the purposes of the present invention, the expression “cationic polymer” denotes any polymer containing cationic groups and/or groups that may be ionized into cationic groups.

The cationic polymers that are preferred are chosen from those containing units comprising primary, secondary, tertiary and/or quaternary amine groups that may either form part of the main polymer chain, or be borne by a side substituent that is directly attached to said chain.

The cationic polymers used generally have a number-average or weight-average molar mass of between 500 and 5×10⁶ approximately and preferably between 10³ and 3×10⁶ approximately.

Among the cationic polymers that may be mentioned more particularly are polymers of the polyamine, polyaminoamide and polyquaternary ammonium type. These are known products.

The polymers of the polyamine, polyaminoamide and polyquaternary ammonium type that may be used in accordance with the present invention, and that may especially be mentioned, are those described in French patents 2 505 348 or 2 542 997. Among these polymers, mention may be made of:

(1) homopolymers or copolymers derived from acrylic or methacrylic esters or amides and comprising at least one of the units of the following formulae:

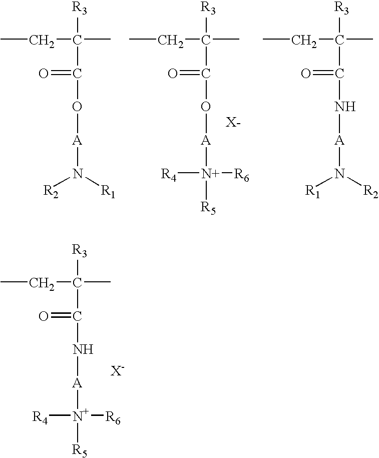

in which:
R₃, which may be identical or different, denote a hydrogen atom or a CH₃ radical;
A, which may be identical or different, represent a linear or branched alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group containing from 1 to 18 carbon atoms or a benzyl radical and preferably an alkyl group containing from 1 to 6 carbon atoms;
R₁ and R₂, which may be identical or different, represent hydrogen or an alkyl group containing from 1 to 6 carbon atoms, and preferably methyl or ethyl;
X denotes an anion derived from an inorganic or organic acid, such as a methosulfate anion or a halide such as chloride or bromide.

Copolymers of family (1) can also contain one or more units derived from comonomers which may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of family (1), mention may be made of:
copolymers of acrylamide and of dimethylaminoethyl methacrylate quaternized with dimethyl sulfate or with a dimethyl halide, such as the product sold under the name Hercofloc by the company Hercules,
the copolymers of acrylamide and of methacryloyloxy-ethyltrimethylammonium chloride described, for example, in patent application EP-A-080 976 and sold under the name Bina Quat P 100 by the company Ciba Geigy,
the copolymers of acrylamide and of methacryloyloxy-ethyltrimethylammonium methosulfate sold under the name Reten by the company Hercules,
quaternized or nonquaternized vinylpyrrolidone/dialkylaminoalkyl acrylate or methacrylate copolymers. These polymers are described in detail in French patents 2 077 143 and 2 393 573,
dimethylaminoethyl methacrylate/vinylcaprolactam/vinylpyrrolidone terpolymers,
vinylpyrrolidone/methacrylamidopropyldimethylamine copolymers, and
quaternized vinylpyrrolidone/dimethylaminopropylmethacrylamide copolymers.

(2) cationic polysaccharides, especially celluloses and cationic galactomannan gums. Among the cationic polysaccharides that may be mentioned more particularly are cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups, which are described in French patent 1 492 597. These polymers are also defined in the CTFA dictionary as hydroxyethylcellulose quaternary ammoniums that have reacted with an epoxide substituted with a trimethylammonium group.

The cationic cellulose copolymers or cellulose derivatives grafted with a water-soluble quaternary ammonium monomer are described especially in U.S. Pat. No. 4,131,576, such as hydroxyalkylcelluloses, for instance hydroxymethyl-, hydroxyethyl- or hydroxypropylcelluloses grafted especially with a methacryloylethyltrimethylammonium, methacrylamidopropyltrimethylammonium or dimethyldiallylammonium salt.

The cationic galactomannan gums are described more particularly in U.S. Pat. Nos. 3,589,578 and 4,031,307, in particular guar gums containing trialkylammonium cationic groups. Use is made, for example, of guar gums modified with a salt (e.g. chloride) of 2,3-epoxypropyltrimethylammonium.

(3) polymers consisting of piperazinyl units and of divalent alkylene or hydroxyalkylene radicals containing straight or branched chains, optionally interrupted by oxygen, sulfur or nitrogen atoms or by aromatic or heterocyclic rings, as well as the oxidation and/or quaternization products of these polymers. Such polymers are described, in particular, in French patents 2 162 025 and 2 280 361;

(4) water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent is used in proportions ranging from 0.025 to 0.35 mol per amine group of the polyamino amide; these polyamino amides can be alkylated or, if they contain one or more tertiary amine functions, they can be quaternized. Such polymers, are described, in particular, in French patents 2 252 840 and 2 368 508;

(5) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical contains from 1 to 4 carbon atoms and preferably denotes methyl, ethyl or propyl. Such polymers are described in particular in French patent 1 583 363.

Among these derivatives, mention may be made more particularly of the adipic acid/dimethylamino-hydroxypropyl/diethylenetriamine polymers sold under the name “Cartaretine F, F4 or F8” by the company Sandoz.

(6) polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyamino amide resulting therefrom is reacted with epichlorohydrin in a molar ratio of epichlorohydrin relative to the secondary amine group of the polyaminoamide of between 0.5:1 and 1.8:1. Such polymers are described in particular in U.S. Pat. Nos. 3,227,615 and 2,961,347.

Polymers of this type are sold in particular under the name “Hercosett 57” by the company Hercules Inc. by the company Hercules in the case of the adipic acid/epoxypropyl/diethylenetriamine copolymer.

(7) cocyclopolymers of alkyldiallylamine or of dialkyldiallylammonium, such as the copolymers containing, as main constituent of the chain, units corresponding to formula (I) or (I′):

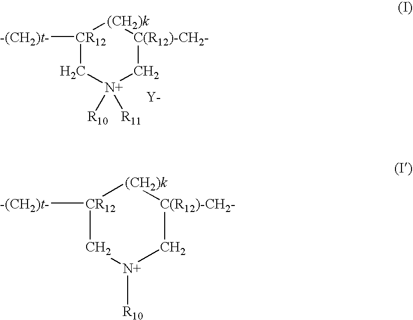

in which formulae k and t are equal to 0 or 1, the sum k+t being equal to 1; R₁₂ denotes a hydrogen atom or a methyl radical; R₁₀ and R₁₁, independently of each other, denote an alkyl group having from 1 to 6 carbon atoms, a hydroxyalkyl group in which the alkyl group preferably has 1 to 5 carbon atoms, a lower C₁-C₄ amidoalkyl group, or R₁₀ and R₁₁ can denote, together with the nitrogen atom to which they are attached, heterocyclic groups such as piperidyl or morpholinyl; Y⁻ is an anion such as bromide, chloride, acetate, borate, citrate, tartrate, bisulfate, bisulfite, sulfate or phosphate. These polymers are described in particular in French patent 2 080 759 and in its Certificate of Addition 2 190 406.

R₁₀ and R₁₁, independently of each other, preferably denote an alkyl group containing from 1 to 4 carbon atoms.

Among the polymers defined above, mention may be made more particularly of the diallyldimethylammonium chloride and acrylamide chloride copolymers sold under the name “Merquat 550” by the company Nalco.

(8) quaternary diammonium polymers containing repeating units corresponding to the formula:

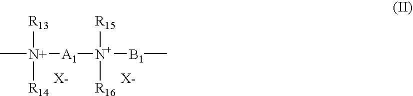

in which formula (II):
R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally containing a second hetero atom other than nitrogen, or alternatively R₁₃, R₁₄, R₁₅ and R₁₆ represent a linear or branched C₁-C₆ alkyl radical substituted with a nitrile, ester, acyl or amide group or a group —CO—O—R₁₇-D or —CO—NH—R₁₇-D where R₁₇ is an alkylene and D is a quaternary ammonium group;
A₁ and B₁ represent polymethylene groups containing from 2 to 20 carbon atoms, which groups may be linear or branched, saturated or unsaturated, and possibly containing, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and
X⁻ denotes an anion derived from an inorganic or organic acid;
A₁, R₁₃ and R₁₅ can form, with the two nitrogen atoms to which they are attached, a piperazine ring; in addition, if A₁ denotes a linear or branched, saturated or unsaturated alkylene or hydroxyalkylene radical, B₁ can also denote a group (CH₂)ₙ—CO-D-OC—(CH₂)ₙ—
in which D denotes:
a) a glycol residue of formula: —O-Z-O—, where Z denotes a linear or branched hydrocarbon-based radical or a group corresponding to one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing an average degree of polymerization;
b) a bis-secondary diamine residue such as a piperazine derivative;
c) a bis-primary diamine residue of formula: —NH—Y—NH—, where Y denotes a linear or branched hydrocarbon-based radical, or alternatively the divalent radical

—CH₂—CH₂—S—S—CH₂—CH₂—;
d) a ureylene group of formula: —NH—CO—NH—;

Preferably, X⁻ is an anion such as chloride or bromide.

These polymers generally have a number-average molecular mass of between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is more particularly possible to use polymers that consist of repeating units corresponding to the formula:

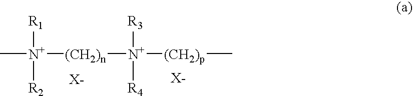

in which R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from an inorganic or organic acid.

One compound of formula (a) that is particularly preferred is the one for which R₁, R₂, R₃ and R₄ represent a methyl radical and n=3, p=6 and X═Cl, which is known as Hexadimethrine chloride according to the INCI (CTFA) nomenclature.

(9) polyquaternary ammonium polymers consisting of units of formula (III):

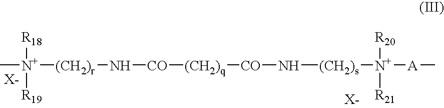

in which formula:
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂ (OCH₂CH₂) pOH radical,
where p is equal to 0 or to an integer between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X⁻ denotes an anion such as a halide,

A denotes a dihalide radical or preferably represents —CH₂—CH₂—O—CH₂—CH₂—

Such compounds are described in particular in patent application EP-A-122 324.

Among these products, mention may be made, for example, of “Mirapol® A 15”, “Mirapol® AD1”, “Mirapol® AZ1” and “Mirapol® 175” sold by the company Miranol.

(10) quaternary polymers of vinylpyrrolidone and of vinylimidazole, such as, for example, the products sold under the names Luviquat® FC 905, FC 550 and FC 370 by the company BASF.

(11) crosslinked methacryloyloxy(C₁-C₄)alkyltri(C₁-C₄)-alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide.

Other cationic polymers that can be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and chitin derivatives, especially chitosans or salts thereof;

The salts that may be used are in particular chitosan acetate, chitosan lactate, chitosan glutamate, chitosan gluconate or chitosan pyrrolidonecarboxylate.

Among these compounds, mention may be made of chitosans with a degree of deacetylation of 90% by weight and chitosan pyrrolidonecarboxylate sold under the name Kytamer® PC by the company Amerchol.

Among all the cationic polymers that may be used in the context of the present invention, it is preferred to use cationic cyclopolymers, in particular the dimethyldiallylammonium chloride homopolymers or copolymers sold under the names “Merquat 100”, “Merquat 550” and “Merquat S” by the company Nalco, quaternary polymers of vinylpyrrolidone and of vinyl-imidazole, crosslinked homopolymers or copolymers of methacryloyloxy (C₁-C₄) alkyltri (C₁-C₄) alkylammonium salts, and chitosan pyrrolidonecarboxylate sold under the name Kytamer® PC by the company Amerchol, and mixtures thereof.

The amphoteric polymers that may be used in accordance with the present invention may be chosen from polymers comprising units K and M randomly distributed in the polymer chain, in which K denotes a unit derived from a monomer comprising at least one basic nitrogen atom and M denotes a unit derived from an acidic monomer comprising one or more carboxylic or sulfonic groups, or alternatively K and M may denote groups derived from zwitterionic carboxybetaine or sulfobetaine monomers; K and M may also denote a cationic polymer chain comprising primary, secondary, tertiary or quaternary amine groups, in which at least one of the amine groups bears a carboxylic or sulfonic group linked via a hydrocarbon-based radical, or alternatively K and M form part of a chain of a polymer containing an α,β-dicarboxylic ethylene unit in which one of the carboxylic groups has been made to react with a polyamine comprising one or more primary or secondary amine groups.

The amphoteric polymers corresponding to the above definition that are more particularly preferred are chosen from the following polymers:

(1) polymers resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylate and acrylate, dialkylaminoalkylmethacrylamide and acrylamide. Such compounds are described in U.S. Pat. No. 3,836,537. Mention may also be made of the sodium acrylate/acrylamidopropyltrimethylammonium chloride copolymer sold under the name Polyquart KE 3033 by the company Cognis.

The vinyl compound may also be a dialkyldiallylammonium salt such as dimethyldiallylammonium chloride. The copolymers of acrylic acid and of the latter monomer are sold under the names Merquat 280 and Merquat 295 by the company Nalco.

(2) polymers containing units derived from:
a) at least one monomer chosen from acrylamides and methacrylamides substituted on the nitrogen with an alkyl radical,
b) at least one acidic comonomer containing one or more reactive carboxylic groups, and
c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides which are more particularly preferred according to the invention are groups in which the alkyl radicals contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen more particularly from acrylic acid, methacrylic acid, crotonic acid, itaconic acid, maleic acid and fumaric acid and alkyl monoesters, having 1 to 4 carbon atoms, of maleic or fumaric acids or anhydrides.

The preferred basic comonomers are aminoethyl, butylaminoethyl, N,N′-dimethylaminoethyl and N-tert-butylaminoethyl methacrylates.

The copolymers whose CTFA (4th edition, 1991) name is Octylacrylamide/acrylates/butylaminoethyl methacrylate copolymer are used more particularly.

(3) crosslinked and alkylated polyamino amides partially or totally derived from polyamino amides of general formula:

CO—R₄—CO-Z (IV)

in which R₄ represents a divalent radical derived from a saturated dicarboxylic acid, a mono- or dicarboxylic aliphatic acid containing an ethylenic double bond, an ester of a lower alkanol, having 1 to 6 carbon atoms, of these acids or a radical derived from the addition of any one of said acids to a bis(primary) or bis(secondary) amine, and Z denotes a bis(primary), mono- or bis(secondary) polyalkylene-polyamine radical and preferably represents:
a) in proportions of from 60 to 100 mol %, the radical

—NH(CH₂)—NHₚ (V)

where x=2 and p=2 or 3, or alternatively x=3 and p=2
this radical being derived from diethylenetriamine, from triethylenetetraamine or from dipropylenetriamine;
b) in proportions of from 0 to 40 mol %, the radical (V) above in which x=2 and p=1 and which is derived from ethylenediamine, or the radical derived from piperazine:

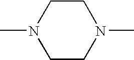

c) in proportions of from 0 to 20 mol %, the —NH—(CH₂)₆—NH— radical derived from hexamethylenediamine, these polyaminoamines being crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyaminoamide and alkylated by the action of acrylic acid, chloroacetic acid or an alkane sultone, or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid and acids containing an ethylenic double bond such as, for example, acrylic acid, methacrylic acid and itaconic acid.

The alkane sultones used in the alkylation are preferably propane sultone or butane sultone, and the salts of the alkylating agents are preferably the sodium or potassium salts.

(4) polymers containing zwitterionic units of formula:

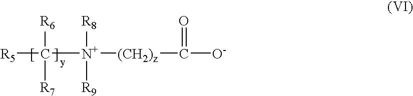

in which R₅ denotes a polymerizable unsaturated group such as an acrylate, methacrylate, acrylamide or methacrylamide group, y and z represent an integer from 1 to 3, R₆ and R₇ represent a hydrogen atom, methyl, ethyl or propyl, R₈ and R₉ represent a hydrogen atom or an alkyl radical such that the sum of the carbon atoms in R₈ and R₉ does not exceed 10.

The polymers comprising such units can also contain units derived from nonzwitterionic monomers such as dimethyl or diethylaminoethyl acrylate or methacrylate or alkyl acrylates or methacrylates, acrylamides or methacrylamides or vinyl acetate.

By way of example, mention may be made of the copolymer of butyl methacrylate/dimethylcarboxymethylammonioethyl methacrylate.

(5) polymers derived from chitosan containing monomer units corresponding to formulae (VII), (VIII) and (IX) below:

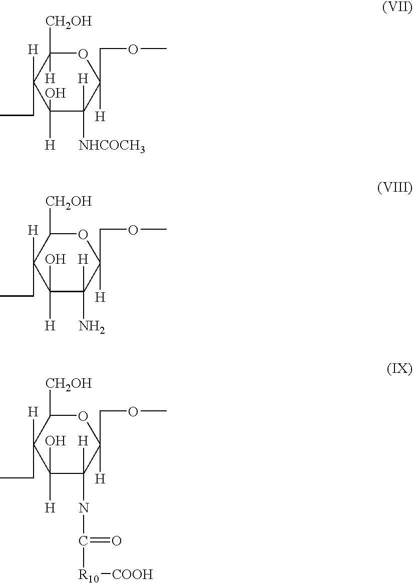

the unit (VII) being present in proportions of between 0 and 30%, the unit (VIII) in proportions of between 5% and 50% and the unit (IX) in proportions of between 30% and 90%, it being understood that, in this unit (IX), R₁₀ represents a radical of formula:

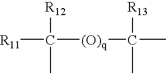

in which
if q=0, R₁₁, R₁₂ and R₁₃, which may be identical or different, each represent a hydrogen atom, a methyl, hydroxyl, acetoxy or amino residue, a monoalkylamine residue or a dialkylamine residue which are optionally interrupted by one or more nitrogen atoms and/or optionally substituted with one or more amine, hydroxyl, carboxyl, alkylthio or sulfonic groups, an alkylthio residue in which the alkyl group bears an amino residue, at least one of the radicals R₁₁, R₁₂ and R₁₃ being, in this case, a hydrogen atom; or, if q=1, R₁₁, R₁₂ and R₁₃ each represent a hydrogen atom, and also the salts formed by these compounds with bases or acids.

(6) polymers derived from the N-carboxyalkylation of chitosan, such as N-carboxymethylchitosan or N-carboxybutylchitosan.

(7) polymers corresponding to the general formula (X) as described, for example, in French patent 1 400 366:

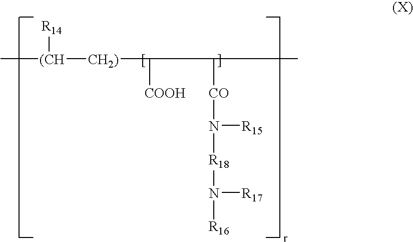

in which R₁₄ represents a hydrogen atom, a CH₃O, CH₃CH₂O or phenyl radical, R₁₅ denotes hydrogen or a lower alkyl radical such as methyl or ethyl, R₁₆ denotes hydrogen or a lower alkyl radical such as methyl or ethyl, R₁₇ denotes a lower alkyl radical such as methyl or ethyl or a radical corresponding to the formula: —R₁₈—N(R₁₆)₂, R₁₈ representing a —CH₂—CH₂—, —CR₂—CH₂—CH₂— or —CH₂—CH(CH₃)— group, R₁₆ having the meanings mentioned above,
and also the higher homologs of these radicals and containing up to 6 carbon atoms.

(8) amphoteric polymers of the type -D-X-D-X- chosen from:
a) polymers obtained by the action of chloroacetic acid or sodium chloroacetate on compounds containing at least one unit of formula:

-D-X-D-X-D- (XI)

where D denotes a radical

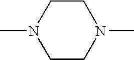

and X denotes the symbol E or E′, E or E′, which may be identical or different, denote a divalent radical which is an alkylene radical with a straight or branched chain containing up to 7 carbon atoms in the main chain, which is unsubstituted or substituted with hydroxyl groups and which can contain, in addition to the oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups;
b) polymers of formula:

-D-X-D-X- (XII)

where D denotes a radical

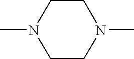

and X denotes the symbol E or E′ and at least once E′; E having the meaning given above and E′ being a divalent radical which is an alkylene radical with a straight or branched chain having up to 7 carbon atoms in the main chain, which is unsubstituted or substituted with one or more hydroxyl radicals and containing one or more nitrogen atoms, the nitrogen atom being substituted with an alkyl chain which is optionally interrupted by an oxygen atom and necessarily containing one or more carboxyl functions or one or more hydroxyl functions and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also contain other vinyl comonomers such as vinylcaprolactam.

The amphoteric polymers that are particularly preferred according to the invention are those of family (1).

The silicones that may be used in accordance with the invention are in particular polyorganosiloxanes that are insoluble in the composition and that may be in the form of oils, waxes, resins or gums.

The organopolysiloxanes are defined in greater detail in Walter Noll's “Chemistry and Technology of Silicones” (1968) Academic Press. They can be volatile or nonvolatile.

When they are volatile, the silicones are more particularly chosen from those having a boiling point of between 60° C. and 260° C., and even more particularly from:

(i) cyclic silicones containing from 3 to 7 and preferably 4 to 5 silicon atoms. These are, for example, octamethylcyclotetrasiloxane sold in particular under the name Volatile Silicone 7207 by Union Carbide or Silbione 70045 V 2 by Rhodia Chimie, decamethylcyclopentasiloxane sold under the name Volatile Silicone 7158 by Union Carbide, and Silbione 70045 V 5 by Rhodia Chimie, and mixtures thereof.

Mention may also be made of cyclocopolymers of the dimethylsiloxanes/methylalkylsiloxane type, such as Volatile. Silicone FZ 3109 sold by the company Union Carbide, having the chemical structure:

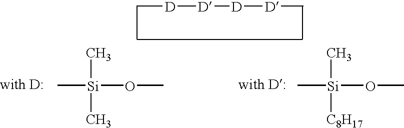

Mention may also be made of mixtures of cyclic silicones with organosilicon compounds, such as the mixture of octamethylcyclotetrasiloxane and tetra-trimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Nonvolatile silicones, and more particularly polyalkylsiloxanes, polyarylsiloxanes, polyalkylarylsiloxanes, silicone gums and resins, polyorganosiloxanes modified with organofunctional groups, and mixtures thereof, are preferably used.

These silicones are more particularly chosen from polyalkylsiloxanes, among which mention may be made mainly of polydimethylsiloxanes containing trimethylsilyl end groups having a viscosity of from 5×10⁻⁶ to 2.5 m²/s at 25° C. and preferably 1×10⁻⁵ to 1 m²/s. The viscosity of the silicones is measured, for example, at 25° C. according to ASTM standard 445 Appendix C.

Among these polyalkylsiloxanes, mention may be made, in a nonlimiting manner, of the following commercial products:
the Silbione oils of the 47 and 70 047 series or the Mirasil oils sold by Rhodia Chimie, such as, for example, the oil 70 047 V 500 000;
the oils of the Mirasil series sold by the company Rhodia Chimie;
the oils of the 200 series from the company Dow Corning, such as, more particularly, DC200 with a viscosity of 0.60 00 cSt;
the Viscasil oils from General Electric and certain oils of the SF series (SF 96, SF 18) from General Electric.

Mention may also be made of polydimethylsiloxanes containing dimethylsilanol end groups (Dimethiconol according to the CTFA name) such as the oils of the 48 series from the company Rhodia Chimie.

In this category of polyalkylsiloxanes, mention may also be made of the products sold under the names Abil Wax 9800 and 9801 by the company Goldschmidt, which are poly(C₁-C₂₀)alkylsiloxanes.

The polyalkylarylsiloxanes are chosen particularly from linear and/or branched polydimethylmethylphenylsiloxanes and polydimethyldiphenylsiloxanes with a viscosity of from 1×10⁻⁵ to 5×10⁻² m²/s at 25° C.

Among these polyalkylarylsiloxanes, mention may be made, by way of example, of the products sold under the following names:
the Silbione oils of the 70 641 series from Rhodia Chimie;
the oils of the Rhodorsil 70 633 and 763 series from Rhodia Chimie;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

The silicone gums that can be used in accordance with the invention are, in particular, polydiorganosiloxanes with high number-average molecular masses of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Mention may be made more particularly of the following products:
polydimethylsiloxane,
polydimethylsiloxane/methylvinylsiloxane gums,
polydimethylsiloxane/diphenylsiloxane,
polydimethylsiloxane/phenylmethylsiloxane,
polydimethylsiloxane/diphenylsiloxane/methylvinylsiloxane.

Products that can be used more particularly in accordance with the invention are mixtures such as:
mixtures formed from a polydimethylsiloxane hydroxylated at the end of the chain (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;
mixtures formed from a polydimethylsiloxane gum with a cyclic silicone, such as the product SF 1214 Silicone Fluid from the company General Electric; this product is an SF 30 gum corresponding to a dimethicone; having a number-average molecular weight of 500 000, dissolved in the oil SF 1202 Silicone Fluid corresponding to decamethylcyclopentasiloxane;
mixtures of two PDMSs of different viscosities, and more particularly of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of ˜5×10⁻⁶ m²/s. This product preferably contains 15% SE 30 gum and 85% SF 96 oil.

The organopolysiloxane resins that can be used in accordance with the invention are crosslinked siloxane systems containing the following units:

R₂SiO_{2/2}, R₃SiO_{1/2}, RSiO_{3/2} and SiO_{4/2} in which R represents a hydrocarbon-based group containing 1 to 16 carbon atoms or a phenyl group. Among these products, those particularly preferred are the ones in which R denotes a C₁-C₄ lower alkyl radical, more particularly methyl, or a phenyl radical.

Among these resins, mention may be made of the product sold under the name Dow Corning 593 or those sold under the names Silicone Fluid SS 4230 and SS 4267 by the company General Electric, which are silicones of dimethyl/trimethylsiloxane structure.

Mention may also be made of the trimethyl siloxysilicate type resins sold in particular under the names X22-4914, X21-5034 and X21-5037 by the company Shin-Etsu.

The organomodified silicones that can be used in accordance with the invention are silicones as defined above and comprising in their structure one or more organofunctional groups attached via a hydrocarbon-based radical.

Among the organomodified silicones, mention may be made of polyorganosiloxanes comprising:
polyethyleneoxy and/or polypropyleneoxy groups optionally comprising C₆-C₂₄ alkyl groups, such as the products known as dimethicone copolyol sold by the company Dow Corning under the name DC 1248 or the oils Silwet L 722, L 7500 μL 77 and L 711 by the company Union Carbide, and the (C₁₋₂)alkylmethicone copolyol sold by the company Dow Corning under the name Q2 5200;
substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C₁-C₄ aminoalkyl groups;
thiol groups such as the products sold under the names GP 72 A and GP 71 from Genesee;
alkoxylated groups such as the product sold under the name Silicone Copolymer F-755 by SWS Silicones and Abil Wax 2428, 2434 and 2440 by the company Goldschmidt;
hydroxylated groups such as the polyorganosiloxanes containing a hydroxyalkyl function, described in French patent application FR-A-85/16334, corresponding to formula (XI):

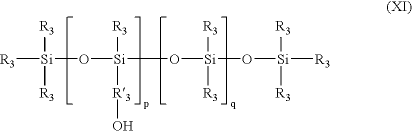

in which the radicals R₃, which may be identical or different, are chosen from methyl and phenyl radicals; at least 60 mol % of the radicals R₃ denoting methyl; the radical R₁₃ is a C₂-C₁₈ divalent hydrocarbon-based alkylene chain unit; p is between 1 and 30 inclusive; q is between 1 and 150 inclusive;
acyloxyalkyl groups such as, for example, the polyorganosiloxanes described in U.S. Pat. No. 4,957,732 and corresponding to formula (XII):

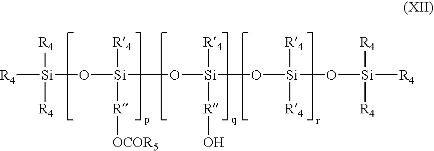

in which:
R₄ denotes a methyl, phenyl, —OCOR₅ or hydroxyl group, one of the radicals R₄ per silicon atom possibly being OH;
R′₄ denotes methyl or phenyl; at least 60 mol % of all the radicals R₄ and R′₄ denoting methyl;
R₅ denotes C₈-C₂₀ alkyl or alkenyl;
R″ denotes a C₂-C₁₈ linear or branched divalent hydrocarbon-based alkylene radical;
r is between 1 and 120 inclusive;
p is between 1 and 30;
q is equal to 0 or is less than 0.5 p, p+q being between 1 and 30; the polyorganosiloxanes of formula (XII) may contain groups:

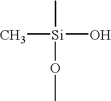

in proportions not exceeding 15% of the sum p+q+r;
anionic groups of carboxylic type, such as, for example, in the products described in patent EP 18.6 507 from the company Chisso Corporation, or of alkylcarboxylic type, such as those present in the product X-22-3701E from the company Shin-Etsu; 2-hydroxyalkylsulfonate; 2-hydroxyalkyl thiosulfate such as the products sold by the company Goldschmidt under the names Abil S201 and Abil S255;
hydroxyacylamino groups, such as the polyorganosiloxanes described in patent application EP 342 834. Mention may be made, for example, of the product Q2-8413 from the company Dow Corning.

According to the invention, it is also possible to use silicones comprising a polysiloxane portion and a portion consisting of a nonsilicone organic chain, one of the two portions constituting the main chain of the polymer, the other being grafted onto said main chain. These polymers are described, for example, in patent applications EP-A-412 704, EP-A-412 707, EP-A-640 105, WO 95/00578, EP-A-582 152 and WO 93/23009 and U.S. Pat. Nos. 4,693,935, 4,728,571 and 4,972,037. These polymers are preferably anionic or nonionic.

Such polymers are, for example, copolymers that can be obtained by free-radical polymerization starting with a monomer mixture consisting of:

a) 50 to 90% by weight of tert-butyl acrylate;
b) 0 to 40% by weight of acrylic acid;
c) 5 to 40% by weight of silicone macromer of formula:

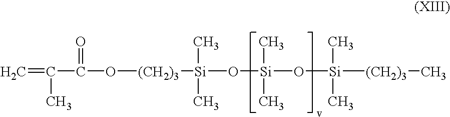

with v being a number ranging from 5 to 700; the weight percentages being calculated relative to the total weight of the monomers.

Other examples of grafted silicone polymers are, in particular, polydimethylsiloxanes (PDMS) onto which are grafted, via a connecting chain unit of thiopropylene type, mixed polymer units of poly(meth)acrylic acid type and of polyalkyl(meth)acrylate type and polydimethylsiloxanes (PDMS) onto which are grafted, via a connecting chain unit of thiopropylene type, polymer units of polyisobutyl(meth)acrylate type.

According to the invention, all of the silicones can also be used in the form of emulsions, nanoemulsions or microemulsions.

The polyorganosiloxanes that are particularly preferred in accordance with the invention are:
nonvolatile silicones chosen from the family of polyalkylsiloxanes containing trimethylsilyl end groups, such as oils having a viscosity of between 0.2 and 2.5 m²/s at 25° C., such as the oils of the DC200 series from Dow Corning, in particular that with a viscosity of 60 000 cSt, of the Silbione 70047 and 47 series and more particularly the oil 70 047 V 500 000, which are sold by the company Rhodia Chimie, polyalkylsiloxanes containing dimethylsilanol end groups, such as dimethiconols, or polyalkylarylsiloxanes such as the oil Silbione 70641 V 200 sold by the company Rhodia Chimie;
the organopolysiloxane resin sold under the name Dow Corning 593;
polysiloxanes containing amine groups, such as amodimethicones or trimethylsilylamodimethicones.

The cationic proteins or cationic protein hydrolyzates are, in particular, chemically modified polypeptides bearing quaternary ammonium groups at the end of the chain or grafted thereto. Their molecular mass can range, for example, from 1500 to 10 000 and in particular from 2000 to 5000 approximately. Among these compounds, mention may be made in particular of:
collagen hydrolyzates bearing triethylammonium groups, such as the products sold under the name Quat-Pro E by the company Maybrook and referred to in the CTFA dictionary as “Triethonium Hydrolyzed Collagen Ethosulfate”;
collagen hydrolyzates bearing trimethylammonium and trimethylstearylammonium chloride groups, sold under the name Quat-Pro S by the company Maybrook and referred to in the CTFA dictionary as “Steartrimonium Hydrolyzed Collagen”;
animal protein hydrolyzates bearing trimethylbenzylammonium groups such as the products sold under the name Crotein BTA by the company Croda and referred to in the CTFA dictionary as “Benzyltrimonium hydrolyzed animal protein”;
protein hydrolyzates bearing, on the polypeptide chain, quaternary ammonium groups containing at least one alkyl radical having from 1 to 18 carbon atoms.

Among these protein hydrolyzates, mention may be made, inter alia, of:
Croquat L in which the quaternary ammonium groups contain a C₁₂ alkyl group;
Croquat M in which the quaternary ammonium groups contain C₁₀-C₁₈ alkyl groups;
Croquat S in which the quaternary ammonium groups contain a C₁₈ alkyl group;
Crotein Q in which the quaternary ammonium groups contain at least one alkyl group having from 1 to 18 carbon atoms.

These various products are sold by the company Croda.

Other quaternized proteins or hydrolyzates are, for example, those corresponding to formula (XIV):

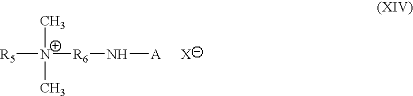

in which X⁻ is an anion of an organic or mineral acid, A denotes a protein residue derived from hydrolyzates of collagen protein, R₅ denotes a lipophilic group containing up to 30 carbon atoms and R₆ represents an alkylene group having 1 to 6 carbon atoms. Mention may be made, for example, of the products sold by the company Inolex under the name Lexein QX 3000, referred to in the CTFA dictionary as “Cocotrimonium Collagen Hydrolyzate”.

Mention may also be made of quaternized plant proteins such as wheat, corn or soybean proteins: as quaternized wheat proteins, mention may be made of those sold, by the company Croda under the names. Hydrotriticum WQ or QM, referred to in the CTFA dictionary as “Cocodimonium Hydrolyzed Wheat Protein”, Hydrotriticum QL, referred to in the CTFA dictionary as “Lauridimonium Hydrolyzed Wheat Protein” or Hydrotriticum QS, referred to in the CTFA dictionary as “Steardimonium Hydrolyzed Wheat Protein”.

According to the present invention, the compounds of ceramide type are in particular natural or synthetic ceramides and/or glycoceramides and/or pseudoceramides and/or neoceramides.

Compounds of ceramide type are described, for example, in patent applications DE 4 424 530, DE 4 424 533, DE 4 402 929, DE 4 420 736, WO 95/23807, WO 94/07844, EP-A-0 646 572, WO 95/16665, FR-2 673 179, EP-A-0 227 994, WO 94/07844, WO 94/24097 and WO 94/10131, the teachings of which are included herein by way of reference.

Compounds of ceramide type that are particularly preferred according to the invention are, for example:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol and in particular N-stearoylphytosphingosine,
2-N-palmitoylaminohexadecane-1,3-diol,
(bis(N-hydroxyethyl-N-cetyl)malonamide),
cetylic acid N-(2-hydroxyethyl)-N-(3-cetyloxy-2-hydroxypropyl)amide,
N-docosanoyl-N-methyl-D-glucamine,
   or mixtures of these compounds.

It is also possible to use cationic surfactants, among which mention may be made in particular of: optionally polyoxyalkylenated primary, secondary or tertiary fatty amine salts; quaternary ammonium salts; imidazoline derivatives; or amine oxides of cationic nature.

Examples of quaternary ammonium salts include:
those of general formula (XV) below:

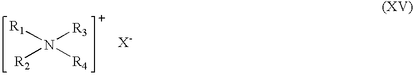

in which the radicals R₁ to R₄, which may be identical or different, represent a linear or branched aliphatic radical containing from 1 to 30 carbon atoms, or an aromatic radical such as aryl or alkylaryl. The aliphatic radicals can comprise heteroatoms such as, in particular, oxygen, nitrogen, sulfur or halogens. The aliphatic radicals are chosen, for example, from alkyl, alkoxy, polyoxy (C₂-C₆) alkylene, alkylamide, (C₁₂-C₂₂)-alkylamido(C₂-C₆)alkyl, (C₁₂-C₂₂)alkylacetate and hydroxyalkyl radicals, comprising from about 1 to 30 carbon atoms; X⁻ is an anion chosen from the group of halides, phosphates, acetates, lactates, (C₂-C₆) alkyl sulfates and alkyl or alkylaryl sulfonates. Preferably, R₁ and R₂ denote C₁-C₄ alkyl or C₁-C₄ hydroxyalkyl.

Among the quaternary ammonium salts of formula (XV) that are preferred are, on the one hand, tetraalkylammonium chlorides, for instance dialkyldimethylammonium or alkyltrimethylammonium chlorides in which the alkyl radical contains from about 12 to 22 carbon atoms, in particular behenyltrimethylammonium chloride, distearyldimethylammonium chloride, cetyltrimethylammonium chloride or benzyldimethylstearylammonium chloride, or, on the other hand, palmitylamidopropyltrimethylammonium chloride or stearamidopropyldimethyl(myristyl acetate)ammonium chloride.
quaternary ammonium salts of imidazolinium, such as, for example, the salt of formula (XVI) below:

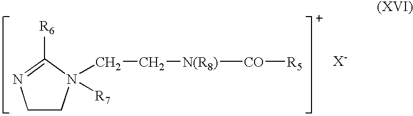

in which R₅ represents an alkenyl or alkyl radical containing from 8 to 30 carbon atoms, for example tallow fatty acid derivatives, R₆ represents a hydrogen atom, a C₁-C₄ alkyl radical or an alkenyl or alkyl radical containing from 8 to 30 carbon atoms, R₇ represents a C₁-C₄ alkyl radical, R₈ represents a hydrogen atom or a C₁-C₄ alkyl radical, X is an anion chosen from the group of halides, phosphates, acetates, lactates, alkyl sulfates and alkyl or alkylaryl sulfonates. R₅ and R₆ preferably denote a mixture of alkenyl or alkyl radicals containing from 12 to 21 carbon atoms, for example tallow fatty acid derivatives, R₇ denotes a methyl radical and R₈ denotes a hydrogen atom. Such a product is sold, for example, under the name “Rewoquat W 75” by the company Degussa;
diquaternary ammonium salts of formula (XVII):

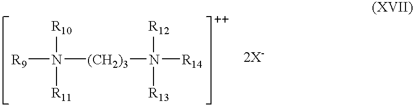

in which R₉ denotes an aliphatic radical containing from about 16 to 30 carbon atoms, R₁₀, R₁₁, R₁₂, R₁₃ and R₁₄, which may be identical or different, are chosen from hydrogen or an alkyl radical containing from 1 to 4 carbon atoms, and X is an anion chosen from the group of halides, acetates, phosphates, nitrates and methyl sulfates. Such diquaternary ammonium salts in particular comprise propane tallow diammonium dichloride;
quaternary ammonium salts containing at least one ester function

The quaternary ammonium salts containing at least one ester function that may be used according to the invention are, for example, those of formula (XVIII) below:

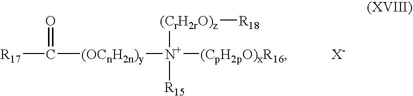

in which:
R₁₅ is chosen from C₁-C₆ alkyl radicals and C₁-C₆ hydroxyalkyl or dihydroxyalkyl radicals;
R₁₆ is chosen from:
a radical

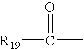

linear or branched, saturated or unsaturated C₁-C₂₂ hydrocarbon-based radicals R₂₀, a hydrogen atom,
R₁₈ is chosen from:
a radical

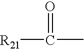

linear or branched, saturated or unsaturated C₁-C₆ hydrocarbon-based radicals R₂₂,
a hydrogen atom,
R₁₇, R₁₉ and R₂₁, which may be identical or different, are chosen from linear or branched, saturated or unsaturated C₇-C₂₁ hydrocarbon-based radicals;
n, p and r, which may be identical or different, are integers ranging from 2 to 6;
y is an integer ranging from 1 to 10;
x and z, which may be identical or different, are integers ranging from 0 to 10;
X⁻ is a simple or complex, organic or inorganic anion;
   with the proviso that the sum x+y+z is from 1 to 15, that when x is 0, then R₁₆ denotes R₂₀ and that when z is 0, then R₁₈ denotes R₂₂.

The R₁₅ alkyl radicals may be linear or branched and more particularly linear.

R₁₅ preferably denotes a methyl, ethyl, hydroxyethyl or dihydroxypropyl radical and more particularly a methyl or ethyl radical.

The sum x+y+z is advantageously from 1 to 10.

When R₁₆ is a hydrocarbon-based radical R₂₀, it may be long and contain from 12 to 22 carbon atoms, or short and contain from 1 to 3 carbon atoms.

When R₁₈ is a hydrocarbon-based radical R₂₂, it preferably contains 1 to 3 carbon atoms.

R₁₇, R₁₉ and R₂₁, which may be identical or different, are advantageously chosen from linear or branched, saturated or unsaturated C₁₁-C₂₁ hydrocarbon-based radicals, and more particularly from linear or branched, saturated or unsaturated, C₁₁-C₂₁ alkyl and alkenyl radicals.

x and z, which may be identical or different, are preferably 0 or 1.

y is advantageously equal to 1.

n, p and r, which may be identical or different, are preferably 2 or 3 and even more particularly are equal to 2.

The anion is preferably a halide (chloride, bromide or iodide) or an alkyl sulfate, more particularly methyl sulfate. However, methanesulfonate, phosphate, nitrate, tosylate, an anion derived from an organic acid, such as acetate or lactate, or any other anion that is compatible with the ammonium containing an ester function, may be used.

The anion X⁻ is even more particularly chloride or methyl sulfate.

The ammonium salts more particularly used are those of formula (XVIII) in which:
R₁₅ denotes a methyl or ethyl radical,
x and y are equal to 1;
z is equal to 0 or 1;
n, p and r are equal to 2;
R₁₆ is chosen from:
   a radical

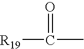

methyl, ethyl or C₁₄-C₂₂ hydrocarbon-based radicals;
a hydrogen atom;
R₁₈ is chosen from:
   a radical

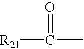

a hydrogen atom;
R₁₇, R₁₉ and R₂₁, which may be identical or different, are chosen from linear or branched, saturated or unsaturated C₁₃-C₁₇ hydrocarbon-based radicals and preferably from linear or branched, saturated or unsaturated C₁₃-C₁₇ alkyl and alkenyl radicals.

The hydrocarbon-based radicals are advantageously linear.

Examples that may be mentioned include the compounds of formula (XVI) such as the diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (chloride or methyl sulfate in particular), and mixtures thereof. The acyl radicals preferably contain 14 to 18 carbon atoms and are obtained more particularly from a plant oil such as palm oil or sunflower oil. When the compound contains several acyl radicals, these radicals may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart by the company Cognis, Stepanquat by the company Stepan, Noxamium by the company CECA or Rewoquat WE 18 by the company Degussa.

It is also possible to use the ammonium salts containing at least one ester function that are described in U.S. Pat. No. 4,874,554 and U.S. Pat. No. 4,137,180.

Among the quaternary ammonium salts of formula (XV), the ones that are preferred are, on the one hand, tetraalkylammonium chlorides such as, for example, dialkyldimethylammonium chlorides or alkyltrimethylammonium chlorides, in which the alkyl radical contains from about 12 to 22 carbon atoms, in particular behenyltrimethylammonium chloride, distearyldimethylammonium chloride, cetyltrimethylammonium chloride, or benzyldimethylstearylammonium chloride, or, on the other hand, stearamidopropyldimethyl (myristyl acetate)-ammonium chloride sold under the name Ceraphyl 70 by the company Van Dyk.

The fatty acids are chosen more particularly from myristic acid, palmitic acid, stearic acid, behenic acid, oleic acid, linoleic acid, linolenic acid and isostearic acid.

The fatty acid derivatives are especially carboxylic acid esters, in particular mono-, di-, tri- or tetracarboxylic esters.

The monocarboxylic acid esters are, in particular, linear or branched, saturated or unsaturated C₁-C₂₆ aliphatic acid monoesters or linear or branched, saturated or unsaturated, C₁-C₂₆ aliphatic alcohols, the total carbon number of these esters being greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isostearyl neopentanoate, isodecyl neopentanoate.

C₄-C₂₂ di- or tricarboxylic acid esters or C₁-C₂₂ alcohols and mono-, di- or tricarboxylic acid esters of C₂-C₂₆ di-, tri-, tetra- or pentahydroxy alcohols can also be used.

Mention may be made in particular of: diethyl sebacate; diisopropyl sebacate; diisopropyl adipate; di-n-propyl adipate; dioctyl adipate; diisostearyl adipate; dioctyl maleate; glyceryl undecylenate; octyldodecylstearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate dicaprate; tridecyl erucate; triisopropyl citrate; triisostearyl citrate; glyceryl trilactate; glyceryl trioctanoate; trioctyldodecyl citrate; trioleyl citrate.

Among the esters mentioned above, it is preferred to use ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate, cetyl octanoate, isostearyl neopentanoate, isodecyl neopentanoate.

The fluoro oils are, for example, the perfluoro-polyethers described in particular in patent application EP-A-486 135 and the fluorohydrocarbon-based compounds described in particular in patent application WO 93/11103. The teaching of these two patent applications is included in its entirety in the present application by way of reference.

The term “fluorohydrocarbon-based compounds” denotes compounds whose chemical structure contains a carbon-based skeleton in which certain hydrogen atoms have been replaced with fluorine atoms.

The fluoro oils can also be fluorocarbons such as fluoroamines, for example perfluorotributylamine, fluorohydrocarbons, for example perfluorodecahydronaphthalene, fluoro esters and fluoro ethers.

The perfluoropolyethers are sold, for example, under the trade names Fomblin by the company Montefluos and Krytox by the company Du Pont.

Among the fluorohydrocarbon-based compounds, mention may also be made of fluorine-containing fatty acid esters such as the product sold under the name Nofable FO by the company Nippon Oil.

Needless to say, it is possible to use mixtures of conditioning agents.

According to the invention, the conditioning agent(s) may represent from 0.001% to 20% by weight, preferably from 0.01% to 10% by weight and more particularly from 0.1% to 3% by weight, relative to the total weight of the final composition.

The composition may comprise a hydrophilic medium comprising water or a mixture of water and of hydrophilic organic solvent(s), for instance alcohols and especially linear or branched C₁-C₆ monoalcohols, for instance ethanol, tert-butanol, n-butanol, isopropanol or n-propanol, and polyols, for instance glycerol, diglycerol, propylene glycol, sorbitol, pentylene glycol and polyethylene glycols, or alternatively glycol ethers, especially C₂ glycol ethers.

The water or the mixture of water and of hydrophilic organic solvents may be present in the composition according to the invention in a content ranging from 30% to 99% by weight and preferably from 40% to 80% by weight relative to the total weight of the composition.

The composition may also comprise an additional polymer such as a film-forming polymer. According to the present invention, the term “film-forming polymer” means a polymer that is capable, by itself or in the presence of an auxiliary film-forming agent, of forming a continuous film that adheres to a support, especially to keratin materials. Among the film-forming polymers that may be used in the composition of the present invention, mention may be made of synthetic polymers, of radical type or of polycondensate type, and polymers of natural origin, and mixtures thereof, in particular acrylic polymers, polyurethanes, polyesters, polyamides, polyureas and cellulose-based polymers, for instance nitrocellulose.

The compositions of the invention also advantageously contain at least one surfactant, which is generally present in an amount of between 0.1% and 60% by weight approximately, preferably between 1% and 40% and even more preferably between 5% and 30%, relative to the total weight of the composition.

This surfactant may be chosen from anionic, amphoteric and nonionic surfactants, or mixtures thereof.

The surfactants that are suitable for carrying out the present invention are especially the following:

### (i) Anionic Surfactant(s):

In the context of the present invention, their nature is not a truly critical factor.

Thus, as examples of anionic surfactants that can be used, alone or as mixtures, in the context of the present invention, mention may be made in particular (nonlimiting list) of salts (in particular alkaline salts, especially sodium salts, ammonium salts, amine salts, amino alcohol salts or magnesium salts) of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates, the alkyl or acyl radical of all of these various compounds preferably containing from 8 to 24 carbon atoms, and the aryl radical preferably denoting a phenyl or benzyl group. Among the anionic surfactants which can also be used, mention may also be made of fatty acid salts such as the salts of oleic, ricinoleic, palmitic and stearic acids, coconut oil acid or hydrogenated coconut oil acid; acyl lactylates in which the acyl radical contains 8 to 20 carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, in particular those containing from 2 to 50 ethylene oxide groups, and mixtures thereof.

Among the anionic surfactants, it is preferred according to the invention to use alkyl sulfate salts and alkyl ether sulfate salts and mixtures thereof.

### (ii) Nonionic Surfactant(s):

The nonionic surfactants are, themselves also, compounds that are well known per se (see in particular in this respect “Handbook of Surfactants” by M. R. Porter, published by Blackie & Son (Glasgow and London), 1991, pp. 116-178) and, in the context of the present invention, their nature is not a critical feature. Thus, they can be chosen in particular from (nonlimiting list) polyethoxylated, polypropoxylated or polyglycerolated fatty acids, fatty alkylphenols, fatty α-diols or fatty alcohols having a fatty chain containing, for example, 8 to 18 carbon atoms, it being possible for the number of ethylene oxide or propylene oxide groups to range in particular from 2 to 50 and for the number of glycerol groups to range in particular from 2 to 30. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. It will be noted that the alkylpolyglycosides constitute nonionic surfactants that are particularly suitable in the context of the present invention.

(iii) Amphoteric Surfactant(s):

The amphoteric surfactants, whose nature is not a critical feature in the context of the present invention, can be, in particular (nonlimiting list), aliphatic secondary or tertiary amine derivatives in which the aliphatic radical is a linear or branched chain containing 8 to 22 carbon atoms and containing at least one water-soluble anionic group (for example carboxylate, sulfonate, sulfate, phosphate or phosphonate); mention may also be made of (C₈-C₂₀)alkylbetaines, sulfobetaines, (C₈-C₂₀)alkylamido (C₁-C₆)alkylbetaines or (C₈-C₂₀) alkylamido (C₁-C₆) alkylsulfobetaines.

Among the amine derivatives, mention may be made of the products sold under the name Miranol, as described in U.S. Pat. Nos. 2,528,378 and 2,781,354 and having the structures:

R₂—CONHCH₂CH₂—N(R₃)(R₄)(CH₂COO—) (2)

in which: R₂ denotes an alkyl radical derived from an acid R₂—COOH present in hydrolyzed coconut oil, a heptyl, nonyl or undecyl radical, R₃ denotes a β-hydroxyethyl group and R₄ denotes a carboxymethyl group;
and

R₅—CONHCH₂CH₂—N(B)(C) (3)

in which:
B represents —CH₂CH₂OX′, C represents —(CH₂)_{z}—Y′ with
z=1 or 2,
X′ denotes the —CH₂CH₂—COOH group or a hydrogen atom,
Y′ denotes —COOH or the —CH₂—CHOH—SO₃H radical,
R₅ denotes an alkyl radical of an acid R₉—COOH present in coconut oil or in hydrolyzed linseed oil, an alkyl radical, in particular a C₇, C₉, C₁₁ or C₁₃ alkyl radical, a C₁₇ alkyl radical and its iso form, an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, mention may be made of the cocoamphodiacetate sold under the trade name Miranol C2M Concentrate by the company Rhodia Chimie.

In the compositions in accordance with the invention, mixtures of surfactants are preferably used, and in particular mixtures of anionic surfactants and mixtures of anionic surfactants and of amphoteric or nonionic surfactants. One mixture that is particularly preferred is a mixture consisting of at least one anionic surfactant and of at least one amphoteric surfactant.

The anionic surfactant preferably used is chosen from sodium, triethanolamine or ammonium (C₁₂-C₁₄)alkyl sulfates, sodium, triethanolamine or ammonium (C₁₂-C₁₄) alkyl ether sulfates oxyethylenated with 2.2 mol of ethylene oxide, sodium cocoyl isethionate and sodium α-(C₁₄-C₁₆)olefin sulfonate, and mixtures thereof, with:
either an amphoteric surfactant such as the amine derivatives known as disodium cocoamphodipropionate or sodium cocoamphopropionate sold especially by the company Rhodia Chimie under the trade name Miranol C2M CONC as an aqueous solution containing 38% active material, or under the name Miranol C32;
or an amphoteric surfactant of zwitterionic type such as alkylbetaines, in particular the cocobetaine sold under the name Dehyton AB 30 as an aqueous solution containing 32% AM by the company Cognis.

Mixtures of surfactants and in particular mixtures of anionic surfactants, mixtures of anionic surfactants and of amphoteric, cationic or nonionic surfactants, and mixtures of cationic surfactants with nonionic or amphoteric surfactants, may be used in the compositions in accordance with the invention. A mixture that is particularly preferred is a mixture comprising at least one anionic surfactant and at least one amphoteric surfactant.

The amount of anionic surfactants preferably ranges from 3% to 40% by weight relative to the total weight of the cosmetic composition. It is preferably between 5% and 35% by weight and better still between 8% and 25% by weight.

When they are present, the amount of amphoteric and/or nonionic surfactants preferably ranges from 0.5% to 20% by weight, in particular from 1% to 15% by weight and more particularly from 2% to 10% by weight relative to the total weight of the composition.

The composition according to the invention may also comprise ingredients commonly used in cosmetics, such as vitamins, fragrances, nacres, thickeners, gelling agents, trace elements, softeners, sequestrants, fragrances, acidifying or basifying agents, preserving agents, sunscreens, antioxidants, hair-loss counteractants, antidandruff agents, grease-controlling agents and free-radical scavengers, or mixtures thereof. Needless to say, a person skilled in the art will take care to select this or these optional additional compound(s), and/or the amount thereof, such that the advantageous properties of the composition according to the invention are not, or are not substantially, adversely affected by the envisioned addition.

Advantageously, the pH of the composition of the present invention is chosen in the range from 2 to 11 and preferentially from 3 to 10.

It is preferably between 3 and 8, and may be adjusted to the desired value by means of acidifying or basifying agents that are well known in the prior art of compositions applied to keratin materials.

Among the basifying agents that may be mentioned, for example, are aqueous ammonia, alkaline carbonates, alkanolamines such as monoethanolmine, diethanolamine and triethanolamine, and derivatives thereof, oxyethylenated and/or oxypropylenated hydroxylalkylamines and ethylenediamines, sodium hydroxide, potassium hydroxide and the compounds of formula (XX) below:

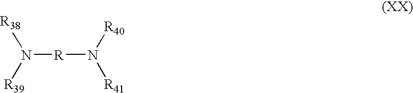

in which R is a propylene residue optionally substituted with a hydroxyl group or a C₁-C₄ alkyl radical; R₃₈, R₃₉, R₄₀ and R₄₁, which may be identical or different, represent a hydrogen atom or a C₁-C₄ alkyl or C₁-C₄ hydroxyalkyl radical.

The acidifying agents are conventionally, by way of example, mineral or organic acids, for instance hydrochloric acid, orthophosphoric acid, carboxylic acids, for instance tartaric acid, citric acid or lactic acid, or sulfonic acids.

The composition according to the invention may comprise a propellant. The propellant is one from among the liquefied or compressed gases usually used for the preparation of aerosol compositions and mixtures thereof. Air, carbon dioxide, compressed nitrogen or a soluble gas such as dimethyl ether, halogenated (in particular fluorinated) hydrocarbons or nonhalogenated hydrocarbons, and mixtures thereof, will preferentially be used.

The compositions essentially find a particularly advantageous application in the haircare field, especially for holding the hair style or shaping the hair. The haircare compositions are preferably shampoos, hair conditioners, hairsetting gels or lotions, blow-drying lotions or fixing and styling compositions such as lacquers or sprays. The lotions may be packaged in various forms, especially in vaporizers, pump-disperser bottles or aerosol containers in order to allow application of the composition in vaporized form or in the form of a mousse.

In another preferred embodiment, the compositions in accordance with the invention may be used for washing or treating keratin materials such as the hair, the skin, the eyelashes, the eyebrows, the nails, the lips or the scalp, and more particularly the hair.

In particular, the compositions according to the invention are detergent compositions such as shampoos, shower gels and bubble baths. In this embodiment of the invention, the compositions comprise one or more surfactants that may be chosen, without preference, alone or as mixtures, from the anionic, amphoteric and nonionic surfactants as defined above.

The quantity and quality of the surfactants are those that are sufficient to give the final composition satisfactory foaming power and/or detergent power.

Thus, according to the invention, the surfactants may represent from 4% to 50% by weight, preferably from 6% to 35% by weight and even more preferentially from 8% to 25% by weight relative to the total weight of the final composition. Preferably, the composition comprises at least 3% by weight and more particularly from 4% to 30% by weight of anionic surfactant relative to the total weight of the composition.

A subject of the invention is thus also a process for treating keratin materials such as the skin or the hair, characterized in that it consists in applying to the keratin materials a cosmetic composition as defined above, and then optionally in rinsing with water.

Thus, this process according to the invention allows hold of the hair style, and treatment, washing or care of or makeup-removal from the skin, the hair or any other keratin material.

A subject of the invention is also the use of the composition for holding or shaping keratin materials, in particular the hair.

In another preferred embodiment, the compositions of the invention may be in the form of a rinse-out or leave-in hair conditioner, or alternatively in the form of rinse-out compositions, to be applied before or after any hair treatment, especially dyeing, bleaching, permanent-waving or relaxing of the hair, or alternatively between the two steps of a permanent-waving or hair-relaxing operation.

When the composition is in the form of a hair conditioner optionally to be rinsed out, it advantageously contains at least one cationic surfactant, for example in a concentration generally of between 0.1% and 10% by weight and preferably from 0.5% to 5% by weight relative to the total weight of the composition.

The compositions according to the invention may also be in the form of aqueous or aqueous-alcoholic lotions for skincare and/or haircare.

The composition according to the invention, after application to human hair and scalp, may be rinsed out or left in after any treatment. It may be in any form conventionally used in the field under consideration, for example in the form of a more or less thickener lotion, a gel, a cream, a spray or a mousse. This composition may be a one-phase or multiphase composition.

The present invention also relates to a cosmetic process for treating keratin materials, which consists in applying an effective amount of a composition as described above to the keratin materials, and optionally rinsing it out after an optional action time.

According to one preferred embodiment of the invention, the composition may be used as a shampoo.

When the compositions in accordance with the invention are used as standard hair conditioners, they are simply applied to wet hair and the composition is then removed, after an optional action time, by rinsing with water, the operation possibly being repeated one or more times.

The compositions of the invention are illustrated in greater detail in the examples that follow.

### Measurement of the Tg

A film is made using an aqueous solution containing 6% by weight of polymer and dried for 48 hours under a controlled atmosphere at 50% relative humidity and 25° C. The films thus obtained have a thickness of between 10 and 20 μm.

The measuring apparatus is a DSC (TA Instruments).

The sample obtained from the film is placed in a hermetic crucible and is heated according to the following protocol:
equilibrium at initial temperature Ti;
heating 1: raising of the temperature, at a rate of +10° C./min to a final temperature: Tf (° C.);
isotherm for 1 minute;
reducing of the temperature at a rate of −10° C./min to Ti (° C.);
heating 2: raising of the temperature at a rate of +10° C./min to Tf (° C.);
isotherm for 1 minute,
   with Ti: initial temperature −120° C.
   with Tf: final temperature+120° C.

The Tg values are measured during the heating steps 1 and 2.

### Measurement of the Water Uptake

About 1 g of dry polymer is placed in an aluminum crucible 4.5 cm in diameter (0.01 m²). It is left to dry for 48 hours in an oven at 60° C. under reduced pressure. The crucibles are removed and weighed immediately (less than one minute after removing from the oven). W1 is obtained.

The crucibles are then placed in a glove box with a given relative humidity (75% HR or 85% HR) and are left therein for 6 hours. They are then weighed again immediately after removing them from the glove box. W2 is obtained.

The water uptake is calculated in the following manner:

[(W2−W1)×100]/W1

### PREPARATION EXAMPLE 1

75 ml of methyl ethyl ketone (MEK) are placed in a reactor (4-necked flask) on which are mounted two addition funnels, a condenser and a mechanical stirrer, and are brought to 80° C.

In parallel, a solution 1 is prepared comprising the monomers: 50 g of polyethylene glycol methacrylate (MPEG 550), 50 g of dimethylaminopropylmethacrylamide (DMAPMA) and the initiator: 0.5 g of (Trigonox 21S). A solution 2 is also prepared, comprising 75 ml of methyl ethyl ketone and 0.5 g of initiator (Trigonox 21S).

Solution 1 is poured dropwise over 1 hour and solution 2 over two hours, into the 4-necked flask reactor. The resulting mixture is then maintained at 80° C. for 5 hours. The orange-yellow solution obtained is cooled 95 g of polymer are obtained.

The polymer has a Brookfield viscosity at 15% in MEK, at 25° C., measured with a No. 1 needle-type spindle, at a speed of 0.1 rpm, of 7.5 mPa·s.

The polymer may then be neutralized in the following manner: 290 ml of 1N HCl are added with stirring to the 0.95 g of polymer and 200 ml of distilled water. The solvent (MEK) is then evaporated off.

The neutralized polymer is soluble in water (at least up to 50% by weight). Its Tg is −60° C.

The neutralized polymer has a water uptake at 85% HR of 51%.

### PREPARATION EXAMPLE 2

100 ml of water are placed in a reactor (4-necked flask) on which are mounted two addition funnels, a condenser and a mechanical stirrer, and are brought to 80° C.

In parallel, a solution 1 comprising 50 g of monomer MPEG 550, 1 g of initiator (potassium persulfate KPS) and 50 ml of water is prepared.

A solution 2 comprising 50 g of monomer DMAPMA 100% neutralized with betaine hydrochloride, and 50 g of water, is also prepared.

Solutions 1 and 2 are poured into the 4-necked flask over 1 hour. After 1 hour at 80° C., a mixture of 1 g of KPS in 50 ml of water is added dropwise thereto over 15 minutes.

The resulting mixture is then maintained at 80° C. for 3 hours. 90 g of polymer neutralized with betaine hydrochloride are obtained.

The polymer has a Brookfield viscosity at 15% in water, at 25° C., measured with a No. 1 needle-type spindle, at a speed of 6 rpm, of 164 mPa·s.

The polymer is soluble in water (at least up to 50% by weight).

Its Tg is −60° C.

The neutralized polymer has a water uptake at 85% HR of 90%.

### PREPARATION EXAMPLES 3 TO 17

The following polymers, which are according to the invention or comparative, are prepared according to the process of example 1 (solvent process) or of example 2 (process in water):

| | | Process | |
|---|---|---|---|
| | | and | |
| | | neutrali- | |
| Example | Monomers | zation | Solubility |
| Example 3 | 10% MPEG 550 | Process 1 | Water |
| | 90% DMAPMA | HCl | |
| Example 4 | 25% MPEG 1100 | Process 1 | Water |
| | 75% DMAPMA | HCl | |
| Example 5 | 50% MPEG 1100 | Process 1 | Water |
| | 50% DMAPMA | HCl | |
| Example 6 | 50% MPEG 550 | Process 1 | Water |
| | 50% DMAPMA | HCl | |
| Example 7 | 50% MPEG 550 | Process 2 | Water |
| | 50% SPE | No | |
| | | neutrali- | |
| | | zation | |
| Example 8 | 50% MPEG 550 | Process 1 | Water |
| | 50% DMAEMA | HCl | |
| Example 9 | 50% MPEG 550 | Process 1 | Water |
| | 50% Morpholinoethyl | HCl | |
| | methacrylate | | |
| Example 10 | 50% MPEG 2000 | Process 2 | Water |
| | 50% DMAPMA | No | |
| | | neutrali- | |
| | | zation | |
| Example 11 | 50% MPEG 550 | Process 1 | Water |
| | 50% DMAPMA | Betaine | |
| | | hydro- | |
| | | chloride | |
| Example 12 | 40% MPEG 550 | Process 1 | Water |
| | 50% DMAPMA | HCl | |
| | 10% EEMA | | |
| Example 13 | 40% MPEG 550 | Process 2 | Water |
| | 50% DMAPMA | HCl | |
| | 10% Hydroxyethyl | | |
| | acrylate | | |
| Example 14 | 40% MPEG 550 | Process 1 | Water |
| | 50% DMAPMA | HCl | |
| | 10% Vinylpyrrolidone | | |
| Example 15 | 40% MPEG 550 | Process 1 | Water |
| | 35% DMAPMA | HCl | |
| | 15% Acrylic acid | | |
| Example 16 | 40% MPEG 550 | Process 1 | Water |
| | 10% | HCl | |
| | Tetrahydrofurfuryl | | |
| | methacrylate | | |
| | 50% DMAPMA | | |
| Example 17 | 40% MPEG 550 | Process 1 | Water |
| | 10% Vinylcaprolactam | HCl | |
| | 50% DMAPMA | | |
| Example 18 | 50% MPEG 550 | Process 1 | Dispersible |
| | 50% DMAPMA | 20% | in water |
| | | Behenic | |
| | | acid | |
| Example 19 | 50% MPEG 550 | Process 1 | Dispersible |
| | 50% DMAPMA | Oleic acid | in water |

MPEG: polyethylene glycol methacrylate (with MW = 550, 1100 or 2000)
DMAPMA: dimethylaminopropylmethacrylamide
SPE: N,N-dimethyl-N-(2-methacryloyloxyethyl)-N-(3-sulfopropyl)ammonium betaine
DMAEMA: dimethylaminoethyl methacrylate
EEMA: ethoxyethyl methacrylate

### Examples of Comparative Polymers

The following polymers not in accordance with the invention are prepared according to example 1:

| | | |
|---|---|---|
| Comparative 1 | 35% MPEG 550 | Process 1 |
| (hydrophobic | 50% DMAPMA | HCl |
| additional | 15% Ethylhexyl acrylate | |
| monomer) | | |
| Comparative 2 | 50% MPEG 550 | Process 1 |
| (crosslinked | 50% DMAPMA | HCl |
| polymer) | 1% Butanediol dimethacrylate | |
| Comparative 3 | 50% MPEG 550 | |
| (quaternized | 50% TMEACL* | |
| polymer) | | |

*TMEACL: 2-(dimethylamino)ethyl acrylate, quaternized with methyl chloride.

### Examples of Compositions

### Examples of Hair Conditioners According to the Invention

The invention may be illustrated with the nonexhaustive compositions that follow. The compositions described below are not limiting. The percentages are expressed as weight percentages of active material.

| | Invention | Invention | Invention |
|---|---|---|---|
| | A | B | C |
| Behentrimonium | 1.2 | 1.2 | 1.2 |
| chloride | | | |
| (Genamin KDMP from | | | |
| Clariant) | | | |
| PEG/PPG | 0.5 | 0.5 | 0.5 |
| Dimethicone | | | |
| (Abil B8851 from | | | |
| Goldschmidt) | | | |
| Cyclopentasiloxane | 15 | 15 | 15 |
| (Dow Corning 245 | | | |
| fluid) | | | |
| Polymer Prep. | 1 | — | — |
| Ex. 11 | | | |
| Polymer Prep. | — | 1.0 | — |
| Ex. 13 | | | |
| Polymer Prep. | — | — | 1.0 |
| Ex. 18 | | | |
| Propylene glycol | 2.5 | 2.5 | 2.5 |
| Preserving agent | qs | qs | qs |
| Fragrance | qs | qs | qs |
| Citric acid/sodium | qs pH 6.5 | qs pH 6.5 | qs pH 6.5 |
| hydroxide | | | |
| Water | qs 100 | qs 100 | qs 100 |

### Examples of Comparative Hair Conditioners

| | D | E | F | G | H |
|---|---|---|---|---|---|
| Behentrimonium | 1.2 | 1.2 | 1.2 | 1.2 | 1.2 |
| chloride | | | | | |
| (Genamin KDMP | | | | | |
| Clariant) | | | | | |
| PEG/PPG | 0.5 | 0.5 | 0.5 | 0.5 | 0.5 |
| Dimethicone | | | | | |
| (Abil B8851 | | | | | |
| Goldschmidt) | | | | | |
| Cyclopenta- | 15 | 15 | 15 | 15 | 15 |
| siloxane | | | | | |
| (Dow Corning | | | | | |
| 245 fluid) | | | | | |
| Polyquaternium-10 | 1 | — | — | — | — |
| (JR400 | | | | | |
| from Rhodia | | | | | |
| Chimie) | | | | | |
| Guar hydroxy | | | | | |
| propyl- | | | | | |
| trimonium | | | | | |
| chloride | | | | | |
| (Jaguar C13S | | | | | |
| from Meyhall) | | | | | |
| Comparative | — | — | 1 | — | — |
| polymer 1 | | | | | |
| Comparative | — | — | — | 1 | — |
| polymer 2 | | | | | |
| Comparative | — | — | — | — | 1 |
| polymer 3 | | | | | |
| Propylene | 2.5 | 2.5 | 2.5 | 2.5 | 2.5 |
| glycol | | | | | |
| Preserving | qs | qs | qs | qs | qs |
| agent | | | | | |
| Fragrance | qs | qs | qs | qs | qs |
| Citric | qs pH | qs pH | qs pH | qs pH | qs pH |
| acid/sodium | 6.5 | 6.5 | 6.5 | 6.5 | 6.5 |
| hydroxide | | | | | |
| Water | qs 100 | qs 100 | qs 100 | qs 100 | qs 100 |

Each composition described above is applied to 10 heads of hair (chestnut-brown European hair about 20 cm long) at a rate of 12 grams of composition per head of hair.

After an action time of 2 minutes, the disentangling on wet hair is evaluated by a panel of 10 experts, and the heads of hair are then rinsed. They are then dried under a hood (50° C./30 minutes).

After drying, the ease of shaping of the heads of hair is evaluated by the same panel of experts (see summary table below).

| | Invention | Invention | Invention | | | | | |
|---|---|---|---|---|---|---|---|---|
| | A | B | C | D | E | F | G | H |
| Disentangling | 7.6 ± 0.4 | 7.1 ± 0.5 | 7.0 ± 0.6 | 8.2 ± 0.7 | 7.8 ± 0.4 | 1.9 ± 0.3 | 2.6 ± 0.5 | 3.6 ± 0.5 |
| on wet hair | | | | | | | | |
| Styling | 6.9 ± 0.5 | 5.3 ± 0.7 | 7.2 ± 0.3 | 2.6 ± 0.4 | 1.7 ± 0.4 | 4.0 ± 0.2 | 4.6 ± 0.4 | 3.1 ± 0.7 |

A grade of 10 corresponds to:
Good disentangling of the hair: average level obtained with the “Masque Intense” hair conditioning formula sold by the company L'Oreal applied at a rate of 12 grams per head of hair (chestnut-brown European hair about 20 cm long). After an action time of 2 minutes, the disentangling on wet hair is evaluated by the panel of experts.

An average level of styling equivalent to that obtained by applying a styling mousse in leave-in mode after blow-drying. 5 g of “Volumax” styling mousse sold by the company L'Oreal are applied to a head of hair (European chestnut-brown hair about 20 cm long). The head of hair is then dried with a hairdryer.

A grade of 0 corresponds to:
Difficult Disentangling
Average level obtained on a head of hair that has just been rinsed.
A Poor Level of Styling.
Average level obtained on a head of hair that has just been rinsed and then dried under a hood (50° C./30 minutes).

The panel of experts systematically indicates that the application of the compositions of the invention (A,B,C) gives a level of styling very much superior to that obtained with the compositions containing the cationic conditioning polymers (D,E).

The panel of experts systematically indicates that the application of the compositions of the invention (A,B,C) gives a level of styling superior to that obtained with the composition containing the polymers with PEG grafts not forming part of the invention (F,G,H).

The panel of experts systematically indicates that the application of the compositions of the invention (A,B,C) gives disentangling of wet hair similar to that obtained with the compositions containing the cationic conditioning polymers (D,E).

The panel of experts systematically indicates that the application of the compositions of the invention (A,B,C) gives disentangling of the hair that is very much greater than that obtained with the composition containing the polymer with PEG grafts not forming part of the invention (F,G,H).

In conclusion, the compositions of the invention (A,B,C) make it possible to obtain a good level of styling while at the same time having a good level of disentangling on wet hair, unlike the comparative compositions (D,E,F,G,H).

### Examples of Shampoo

| | Ex 1 | Ex 2 | Ex 3 | Ex 4 |
|---|---|---|---|---|
| SODIUM LAURETH | 12% AM | 12% AM | 12% AM | 12% AM |
| SULFATE [1] | | | | |
| COCOBETAINE [3] | — | 2% AM | 2% AM | — |
| COCOAMIDOPROPYL | 2% AM | — | — | 2% AM |
| BETAINE [4] | | | | |
| POLYQUATERNIUM | 0.5% AM | — | — | 0.5% AM |
| 10 [6] | | | | |
| DMAPMA/MPEG 550 | 0.5% AM | 0.5% AM | 0.5% AM | 0.5% AM |
| (50/50) [7] | | | | |
| GUAR | — | 0.1% AM | — | — |
| HYDROXYPROPYL- | | | | |
| TRIMONIUM | | | | |
| CHLORIDE [8] | | | | |
| DIMETHICONE [9] | — | 2% AM | — | — |
| DIMETHICONE [10] | — | — | 2% AM | 1.0% AM |
| COCOAMIDE | — | — | 1.5% AM | 1.5% AM |
| MIPA [13] | | | | |
| PRESERVING AGENT | qs | qs | qs | qs |
| FRAGRANCE | qs | qs | qs | qs |
| SODIUM | qs pH 6 | qs pH 7 | qs pH 7 | qs pH 7 |
| HYDROXIDE/CITRIC | | | | |
| ACID | | | | |
| WATER | qs 100% | qs 100% | qs 100% | qs 100% |

| | | Ex 5 | Ex 6 | Ex 7 |
|---|---|---|---|---|
| SODIUM LAURETH | | 12% AM | 12% AM | 12% AM |
| SULFATE [1] | | | | |
| COCOBETAINE [3] | | — | 2% AM | 2% AM |
| DISODIUM COCOAMPHO- | | 2% AM | — | — |
| DIACETATE [5] | | | | |
| DMAPMA/MPEG 550 | | 0.5% AM | 0.5% AM | 0.5% AM |
| (50/50) [7] | | | | |
| GUAR HYDROXYPROPYL- | | 0.2% AM | 0.2% AM | — |
| TRIMONIUM | | | | |
| CHLORIDE [8] | | | | |
| DIMETHICONE [11] | | 2.0% AM | — | — |
| AMODIMETHICONE [12] | | — | 2% AM | 3% AM |
| COCOAMIDE MIPA [13] | | 1.5% AM | 1.5% AM | 1.5% AM |
| PRESERVING AGENT | | Qs | Qs | Qs |
| FRAGRANCE | | Qs | Qs | Qs |
| SODIUM HYDROXIDE/ | | qs pH 7 | qs pH 7 | qs pH 7 |
| CITRIC ACID | | | | |
| WATER | | qs 100% | qs 100% | qs 100% |
| | Ex 9 | Ex 10 | Ex 11 | Ex 12 |
| SODIUM | 8% AM | 8% AM | 8% AM | 8% AM |
| LAURETH | | | | |
| SULFATE [1] | | | | |
| COCO- | 5% AM | 5% AM | 5% AM | 5% AM |
| GLUCOSIDE [2] | | | | |
| COCO- | 2% AM | 2% AM | 2% AM | 2% AM |
| BETAINE [3] | | | | |
| POLY- | 0.5% AM | — | — | 0.5% AM |
| QUATERNIUM | | | | |
| 10 [6] | | | | |
| DMAPMA/ | 0.5% AM | 0.5% AM | 0.5% AM | 0.5% AM |
| MPEG 550 | | | | |
| (50/50) [7] | | | | |
| GUAR | — | 0.1% AM | — | — |
| HYDROXY- | | | | |
| PROPYL- | | | | |
| TRIMONIUM- | | | | |
| CHLORIDE [8] | | | | |
| DIMETH- | — | 2% AM | — | — |
| ICONE [9] | | | | |
| DIMETH- | — | — | 2% AM | 1.0% AM |
| ICONE [10] | | | | |
| COCOAMIDE | — | — | 1.5% AM | 1.5% AM |
| MIPA [13] | | | | |
| PRESERVING | Qs | Qs | Qs | Qs |
| AGENT | | | | |
| FRAGRANCE | Qs | Qs | Qs | Qs |
| SODIUM | Qs pH 6 | Qs pH 7 | Qs pH 7 | Qs pH 7 |
| HYDROXIDE/ | | | | |
| CITRIC ACID | | | | |
| WATER | Qs 100% | Qs 100% | Qs 100% | Qs 100% |

| | Ex 13 | Ex 14 | Ex 15 |
|---|---|---|---|
| SODIUM LAURETH | 8% AM | 8% AM | 8% AM |
| SULFATE [1] | | | |
| COCO-GLUCOSIDE [2] | 5% AM | 5% AM | 5% AM |
| COCOBETAINE [3] | 2% AM | 2% AM | 2% AM |
| POLYQUATERNIUM | — | — | — |
| 10 [6] | | | |
| DMAPMA/MPEG 550 | 0.5% AM | 0.5% AM | 0.5% AM |
| (50/50) [7] | | | |
| GUAR | 0.2% AM | 0.2% AM | — |
| HYDROXYPROPYL- | | | |
| TRIMONIUM | | | |
| CHLORIDE [8] | | | |
| DIMETHICONE [11] | 2% AM | — | — |
| AMODIMETHICONE [12] | — | 2% AM | 3% AM |
| COCOAMIDE MIPA [13] | 1.5% AM | 1.5% AM | 1.5% AM |
| PRESERVING AGENT | qs | qs | qs |
| FRAGRANCE | qs | qs | qs |
| SODIUM | qs pH 7 | qs pH 7 | qs pH 7 |
| HYDROXIDE/ | | | |
| CITRIC ACID | | | |
| WATER | qs 100% | qs 100% | qs 100% |

[1] Texapon N 702 (Cognis)
[2] Plantacare 818 UP (Cognis)
[3] Dehyton AB 30 (Cognis)
[4] Tego betaine F50 (Goldschmidt)
[5] Miranol C 2M- conc. NP (Rhodia)
[6] Ucar polymer JR400 LT (Amerchol)
[7] Polymer of example 11
[8] Jaguar C13S (Rhodia)
[9] Belsil DM 300 000 (Wacker)
[10] Mirasil DM 500 000 (Rhodia)
[11] Dow Corning 200 fluid 60 000 (Dow Corning)
[12] Dow Corning 939 emulsion (Dow Corning)
[13] Empilan CIS (Huntsman)

### Examples of Hair Conditioner According to the Invention

| | 16 | |
|---|---|---|
| a,ω-polydimethylsiloxane | 0.25 | g AM |
| quaternary ammonium acetate | | |
| containing a coconut chain | | |
| (Quaternium-80, Abil Quat | | |
| 3272 from Goldschmidt) | | |
| Hydroxypropyl starch | 5.3 | g AM |
| phosphate | | |
| PEG-14 dimethicone | 0.25 | g |
| (Abil B8842 from Goldschmidt) | | |
| Hydroxyethylcellulose | 0.4 | g |
| Polymer Prep. Ex. 11 | 1.5 | g |
| Aminomethylpropanol | 1.5 | g |
| Citric acid | 3 | |
| Preserving agent | qs | |
| Fragrance | qs | |
| Citric acid/sodium hydroxide | qs pH 6.5 | |
| Water | qs 100 | g |

1. A cosmetic composition, characterized in that it comprises, in a cosmetically acceptable aqueous medium:
I) at least one conditioning agent chosen from synthetic oils, mineral oils, plant oils, fluoro or perfluoro oils, natural or synthetic waxes, silicones, cationic polymers with a cationic charge density of less than 5 meq/g, cationic proteins, cationic protein hydrolyzates, compounds of ceramide type, cationic surfactants, fatty amines, fatty acids and derivatives thereof, and also mixtures of these various compounds, and
II) at least one ethylenic copolymer comprising, as a weight percentage relative to the total weight of the polymer:
a) 10-60% by weight of one or more monomers of formula (I): 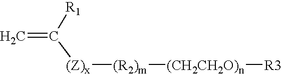
in which:
R1 is a hydrogen atom or a linear or branched hydrocarbon-based radical, of the type CₚH₂ₚ₊₁, with p being an integer between 1 and 12 inclusive;
Z is a divalent group chosen from —COO—, —CONH—, —CONCH₃—, —OCO—, —O—, —SO₂—, —CO—O—CO— and —CO—CH₂—CO—;
x is 0 or 1;
R2 is a saturated or unsaturated, optionally aromatic, linear, branched or cyclic carbon-based divalent radical of 1 to 30 carbon atoms, possibly comprising 1 to 18 heteroatoms chosen from O, N, S, F, Si and P;
m is 0 or 1;
n is an integer between 3 and 300 inclusive;
R3 is a hydrogen atom or a saturated or unsaturated, optionally aromatic, linear, branched or cyclic carbon-based radical of 1 to 30 carbon atoms, possibly comprising 1 to 20 heteroatoms chosen from O, N, S, F, Si and P;
and salts thereof;
b) 40-90% by weight of at least one “essentially cationic” monomer or a salt thereof, chosen from:
(i) one or more cationic monomers of formula (IIa),
(ii) one or more amphoteric monomers of formulae (IIc) and (IId), and
(iii) a mixture of one or more cationic monomers of formula (IIa) with one or more anionic monomers chosen from maleic anhydride and/or those of formula (IIb); and/or with one or more amphoteric monomers chosen from those of formulae (IIc) and (IId); 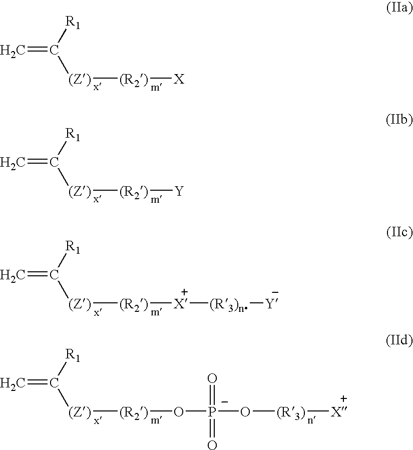
in which:
R1 is a hydrogen atom or a linear or branched hydrocarbon-based radical of the type CₚH₂ₚ₊₁, with p being an integer between 0.1 and 12 inclusive; preferably hydrogen or a methyl, ethyl, propyl or butyl radical.
Z′ is a divalent group chosen from —COO—, —CONH—, —CONCH₃—, —OCO— or —O—, —SO₂— —CO—O—CO— or —CO—CH₂—CO—; preferably, COO and CONH.
x′ is 0 or 1, preferably 1.
R′2 is a saturated or unsaturated, optionally aromatic, linear, branched or cyclic divalent carbon-based radical of 1 to 30 carbon atoms, possibly comprising 1 to 18 heteroatoms chosen from O, N, S, F, Si and P;
m′ is 0 or 1;
X (in formula IIa) is (a) a group of formula —N(R₆)(R₇) or —P(R₆)(R₇) or —P⁺R₆R₇R₈, with R6, R7 and R8 representing, independently of each other, either (i) a hydrogen atom, or (ii) a linear, branched or cyclic, saturated or unsaturated, optionally aromatic alkyl group containing from 1 to 18 carbon atoms, possibly comprising 1 to 10 heteroatoms chosen from O, N, S, F, Si and P; or (iii) R6 and R7 may form with the nitrogen or phosphorus atom a first saturated or unsaturated, optionally aromatic ring comprising in total 5, 6, 7 or 8 atoms, and especially 4, 5 or 6 carbon atoms and/or 2 to 4 heteroatoms chosen from O, S and N; said first ring possibly being fused with one or more other saturated or unsaturated, optionally aromatic rings each comprising 5, 6 or 7 atoms, and especially 4, 5, 6 or 7 carbon atoms and/or 2 to 4 heteroatoms chosen from O, S and N;
or (b) X represents a group —R′6-N—R′7- in which R′6 and R17 form with the nitrogen atom a saturated or unsaturated, optionally aromatic ring comprising in total 5, 6, 7 or 8 atoms, and especially 4, 5 or 6 carbon atoms and/or 2 to 4 heteroatoms chosen from O, S and N; said ring possibly being fused with one or more other saturated or unsaturated, optionally aromatic rings, each comprising 5, 6 or 7 atoms, and especially 4, 5, 6, 7 or 8 carbon atoms and/or 2 to 4 heteroatoms chosen from O, S and N;
Y is a group chosen from —COOH, —SO₃H, —OSO₃H, —PO₃H₂ and —OPO₃H₂;
X′⁺ is a divalent group of formula —N⁺(R₆)(R₇)— with R6 and R₇ representing, independently of each other, either (i) a hydrogen atom, or (ii) a linear, branched or cyclic, optionally aromatic alkyl group containing from 1 to 25 carbon atoms, possibly comprising 1 to 20 heteroatoms chosen from O, N, S and P; or (iii) R6 and R7 may form with the nitrogen atom a first saturated or unsaturated, optionally aromatic ring comprising in total 5, 6, 7 or 8 atoms, and especially 4, 5, 6 or 7 carbon atoms and/or 2 to 3 heteroatoms chosen from O, S and N; said first cycle possibly being fused with one or more other saturated or unsaturated, optionally aromatic rings, each comprising 5, 6, 7 or 8 atoms, and especially 4, 5, 6 or 7 carbon atoms and/or 2 to 3 heteroatoms chosen from O, S and N;
Y′⁻ is a group chosen from —COO⁻, —SO₃, —OSO₃⁻, —PO₃² and —OPO₃²⁻;
R′3 is a saturated or unsaturated, optionally aromatic, linear, branched or cyclic divalent carbon-based radical of 1 to 30 carbon atoms, possibly comprising 1 to 18 heteroatoms chosen from O, N, S, F, Si and P;
n′ is between 1 and 100 and preferably between 1 and 5 inclusive;
X″⁺ is a group of formula —N⁺R₆R₇R₈ with R6, R7 and R8 representing, independently of each other, either (i) a hydrogen atom, or (ii) a linear, branched or cyclic, optionally aromatic alkyl group containing from 1 to 18 carbon atoms, possibly comprising 1 to 5 heteroatoms chosen from O, N, S and P; or (iii) R6 and R7 may form with the nitrogen atom a first saturated or unsaturated, optionally aromatic ring comprising in total 5, 6 or 7 atoms, and especially 4, 5 or 6 carbon atoms and/or 2 to 3 heteroatoms chosen from O, S and N; said first ring possibly being fused with one or more other saturated or unsaturated, optionally aromatic rings, each comprising 5, 6 or 7 atoms, and especially 4, 5, 6 or 7 carbon atoms and/or 2 to 3 heteroatoms chosen from O, S and N;
c) and optionally 0-50% by weight of nonionic hydrophilic monomers, excluding methyl acrylate, methyl methacrylate and isopropyl acrylate if they are present in an amount of greater than or equal to 10% by weight.

2. The composition as claimed in claim 1, characterized in that, in formula (I), R1 represents hydrogen or a methyl, ethyl, propyl or butyl radical.

3. The composition as claimed in either of the preceding claims, characterized in that, in formula (I), Z represents COO or CONH.

4. The composition as claimed in one of the preceding claims, characterized in that, in formula (I), the radical R2 is chosen from:
an alkylene radical such as methylene, ethylene, propylene, n-butylene, isobutylene, tert-butylene, n-hexylene, n-octylene, n-dodecylene, n-octadecylene, n-tetradecylene or n-docosanylene;
a phenylene radical —C₆H₄— (ortho, meta or para), optionally substituted with a C1-C12 alkyl radical optionally comprising 1 to 25 heteroatoms chosen from O, N, S, F, Si and P; or alternatively a benzylene radical —C₆H₄—CH₂—, optionally substituted with a C1-C12 alkyl radical optionally comprising 1 to 8 heteroatoms chosen from O, N, S, F, Si and P;
a pyridinium radical of formula: 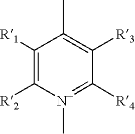
with R′1 to R′4, which may be identical or different, chosen from H and a C1-C12 alkyl radical optionally comprising 1 to 8 heteroatoms chosen from O, N, S, F, Si and P; R′1 to R′4 may especially be methyl and/or ethyl;
a radical of formula —CH₂—CHOH—, —CH₂—CH₂—CHOH—, CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, —CH₂—CH₂—CH₂—NR′—, —CH₂—O—CO—O—, CH₂—CH₂—O—CO—O—, —CH₂—CO—O—, —CH₂—CH₂—CO—O—, —CH₂—O—CO—NH—, —CH₂—CH₂—O—CO—NH—; —CH₂—NH—CO—NH— or —CH₂—CH₂—NH—CO—NH—; —CH₂—CH₂—CH₂—O—; —CH₂—CH₂—CHR′—O— with R′ and R″ representing a linear or branched C1-C22 alkyl optionally comprising 1 to 12 heteroatoms chosen from O, N, S, F, Si and P;
or a mixture of these radicals.

5. The composition as claimed in one of the preceding claims, characterized in that, in formula (I), n is between 5 and 200 inclusive and better still between 7 and 100 inclusive, or even between 9 and 50 inclusive.

6. The composition as claimed in one of the preceding claims, characterized in that, in formula (I), R3 is a hydrogen atom; a succinimido, maleimido, mesityl, tosyl, triethoxysilane, phthalimide or —CH₂—CH₂CN radical; or alternatively a benzyl or phenyl radical optionally substituted with a C1-C12 alkyl radical optionally comprising 1 to 8 heteroatoms chosen from O, N, S, F, Si and P; a C1-C30 and especially C1-C22 or even C2-C16 alkyl radical, optionally comprising 1 to 18 heteroatoms chosen from O, N, S, F, Si and P;
said benzyl, phenyl or alkyl radicals also possibly comprising a function chosen from the following functions: succinimido; glutarate-succinimido; glutarate; maleimido; mesityl, benzoate; tosyl; triethoxysilane; phthalimide; thioester; benzotriazole carbonate; butyraldehyde; acetaldehyde diethyl acetal; biotin; phospholipid; succinate; N-hydroxysuccinimide; —SO₃H, —COOH, —PO₄, —NR5R6 or —N⁺R5R6R7, with R5, R6 and R7, independently of each other, chosen from H and linear, branched or cyclic C1-C18 alkyls, especially methyl, optionally comprising one or more heteroatoms or alternatively protecting groups such as t-butyloxycarbonyl or 9-fluorenylmethoxycarbonyl.

7. The composition as claimed in one of the preceding claims, characterized in that the monomer of formula (I) is chosen, alone or as a mixture, from:
poly(ethylene glycol) (meth)acrylate;
methylpoly(ethylene glycol) (meth)acrylate;
alkylpoly(ethylene glycol) (meth)acrylates;
phenylpoly(ethylene glycol) (meth)acrylates;
the following monomer: 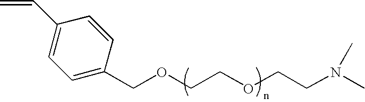
characterized in that n is preferably between 3 and 100 inclusive and especially 5 to 50 inclusive, or even 7 to 30 inclusive.

8. The composition as claimed in one of the preceding claims, characterized in that the monomer of formula (I), alone or as a mixture, is present in a proportion of from 20% to 55% by weight and preferably from 30% to 50% by weight relative to the weight of the final polymer.

9. The composition as claimed in one of the preceding claims, characterized in that, in formulae (IIa), (IIb), (IIc) and/or (IId), the radical R′2 is chosen from:
an alkylene radical such as methylene, ethylene, propylene, n-butylene, isobutylene, tert-butylene, n-hexylene, n-octylene, n-dodecylene, n-octadecylene, n-tetradecylene or n-docosanylene;
a phenylene radical —C₆H₄— (ortho, meta or para), optionally substituted with a C1-C12 alkyl radical optionally comprising 1 to 5 heteroatoms chosen from N, O, S, F, Si and/or P; or alternatively a benzylene radical —C₆H₄—CH₂—, optionally substituted with a C1-C12 alkyl radical optionally comprising 1 to 5 heteroatoms chosen from O, N, S, F, Si and P;
a radical of formula —CH₂—O—CO—O—, CH₂—CH₂'O—CO—O—, —CH₂—CO—O—, —CH₂—CH₂—CO—O—, —[(CH₂)₅—CO—O]ₙ—, —CH₂—CH(CH₃)—, —(CH₂)₂—O—CH₂—O—CO—NH—, —CH₂—CH₂—O—CO—NH—; —CH₂—NH—CO—NH— or —CH₂—CH₂—NH—CO—NH—, —CH₂—CHOR—, —CH₂—CH₂—CHOH—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, —CH₂—CH₂—CH₂—NR′—, —CH₂—CH₂—CH₂—O—; —CH₂—CH₂—CHR′—O— with R′ and R″ representing a linear or branched C1-C22 alkyl optionally comprising 1 to 12 heteroatoms chosen from O, N, S, F, Si and P;
or a mixture of these radicals.

10. The composition as claimed in one of the preceding claims, characterized in that, in formula (IIa), the radicals R6 and R7 present in X are chosen from hydrogen and a methyl, ethyl, propyl, isopropyl, n-butyl, t-butyl, isobutyl, octyl, lauryl or stearyl group.

11. The composition as claimed in one of the preceding claims, characterized in that, in formula (IIa), X is a radical chosen from radicals of pyridine, indolyl, isoindolinyl, imidazolyl, imidazolinyl, piperidyl, pyrazolinyl, pyrazolyl, quinoline, pyrazolinyl, pyridyl, piperazinyl, pyrrolidinyl, quinidinyl, thiazolinyl, morpholine, guanidino, amidino or phosphonium type, and mixtures thereof.

12. The composition as claimed in one of the preceding claims, characterized in that the monomers of formula (IIa) are neutralized with neutralizers chosen from neutralizers with a log P of less than or equal to 2, for example between −8 and 2, preferably between −6 and 1 and especially between −6 and 0; and/or with agents with a log P of greater than 2, preferably greater than or equal to 2.5, especially greater than 3, and in particular between 3 and 15, or even between 3.5 and 10.

13. The composition as claimed in one of the preceding claims, characterized in that the monomer of formula (IIa) is chosen, alone or as a mixture, from: dimethylaminopropyl(meth)acrylamide, dimethylaminoethyl(meth)acrylamide, diethylaminoethyl(meth)acrylate, dimethylaminoethyl(meth)acrylate, vinylimidazole, vinylpyridine and morpholinoethyl(meth)acrylate, and the monomers below:

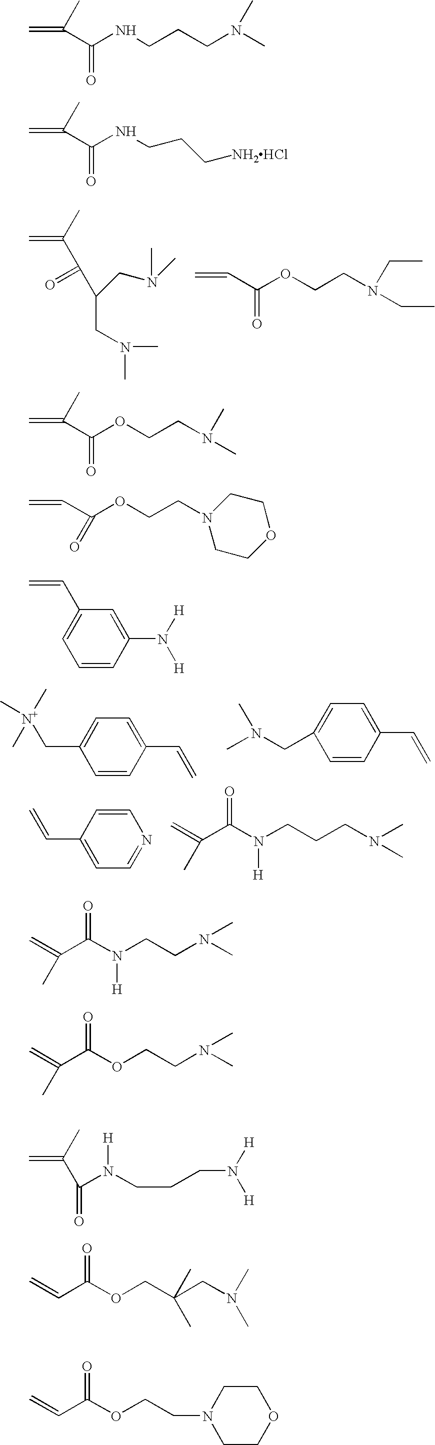

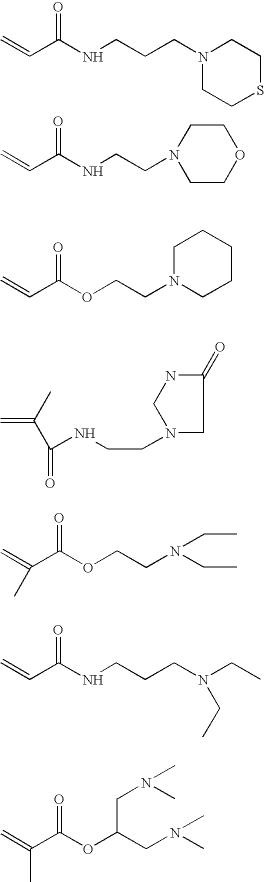

14. The composition as claimed in one of the preceding claims, characterized in that the anionic monomers are chosen from maleic anhydride, acrylic acid, methacrylic acid, crotonic acid, itaconic acid, fumaric acid, maleic acid, 2-carboxyethyl acrylate (CH₂═CH—C(O)—O—(CH₂)₂—COOH); styrenesulfonic acid, 2-acrylamido-2-methylpropanesulfonic acid, vinylbenzoic acid, vinylphosphoric acid and sulfopropyl (meth)acrylate, and the salts thereof.

15. The composition as claimed in one of the preceding claims, characterized in that, in formula (IIc), the radical X′⁺ is chosen from the radicals of pyridine, indolyl, isoindolinyl, imidazolyl, imidazolinyl, piperidyl, pyrazolinyl, pyrazolyl, quinoline, pyrazolinyl, pyridyl, piperazinyl, pyrrolidinyl, quinidinyl, thiazolinyl, morpholine, guanidino and amidino type, and mixtures thereof.

16. The composition as claimed in one of the preceding claims, characterized in that, in formulae (IIc) and/or (IId), the radical R′3 is chosen from:
an alkylene radical such as methylene, ethylene, propylene, n-butylene, isobutylene, tert-butylene, n-hexylene, n-octylene, n-dodecylene, n-octadecylene, n-tetradecylene or n-docosanylene;
a phenylene radical —C₆H₄— (ortho, meta or para), optionally substituted with a C1-C12 alkyl radical optionally comprising 1 to 5 heteroatoms chosen from O, N, S, F, Si and P; or alternatively a benzylene radical —C₆H₄—CH₂—, optionally substituted with a C1-C12 alkyl radical optionally comprising 1 to 5 heteroatoms chosen from O, N, S, F, Si and P;
a radical of formula —CH₂—O—CO—O—, CH₂—CH₂—O—CO—O—, —CH₂—CO—O—, —CH₂—CH₂—CO—O—, —[(CH₂)₅—CO—O]ₙ—, —CH₂—CH(CH₃)—O—, —(CH₂)₂—O—, —CH₂—O—CO—NH—, —CH₂—CH₂—O—CO—NH—; —CH₂—NH—CO—NH— or —CH₂—CH₂—NH—CO—NH—, —CH₂—CHOH—, —CH₂—CH₂—CHOH—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, —CH₂—CH₂—CH₂—NR′—, —CH₂—CH₂—CH₂—; —[CH₂—CH₂—O]ₙ— and —[CH₂—CH(CH₃)—O]ₙ—, —CH₂—CH₂—CHR′—O— with R′ and R″ representing a linear or branched C1-C22 alkyl optionally comprising 1 to 12 heteroatoms chosen from O, N, S, F, Si and P;
or a mixture of these radicals.

17. The composition as claimed in one of the preceding claims, characterized in that, in formula (IId), X″⁺ is chosen from trimethylammonium; triethylammonium; N,N-dimethyl-N-octylammonium; N,N-dimethyl-N-laurylammonium radicals.

18. The composition as claimed in one of the preceding claims, characterized in that the monomer of formula (IIc) or (IId) is chosen from N,N-dimethyl-N-(2-methacryloyloxyethyl)-N-(3-sulfopropyl)ammonium betaine, N,N-dimethyl-N-(3-methacrylamidopropyl)-N-(3-sulfopropyl)ammonium betaine, 1-(3-sulfopropyl)-2-vinylpyridinium betaine and 2-methacryloyloxyethylphosphorylcholine.

19. The composition as claimed in one of the preceding claims, characterized in that, when the “essentially cationic” monomer is chosen from mixtures of cationic and/or amphoteric monomers with anionic monomers, said anionic monomers are preferably present in a proportion of from 5% to 40% by weight, especially from 10% to 30% by weight and preferably from 15% to 25% by weight relative to the weight of the “cationic and/or amphoteric+anionic” mixture.

20. The composition as claimed in one of the preceding claims, characterized in that the “essentially cationic” monomer is present in a proportion of from 45% to 80% by weight and preferably from 50% to 70% by weight relative to the weight of the final polymer.

21. The composition as claimed in one of the preceding claims, characterized in that the additional nonionic hydrophilic monomer has a log P of between −8 and 2, preferably less than or equal to 1.5, especially less than or equal to 1 and in particular between −7 and 1, or even between −6 and 0.

22. The composition as claimed in one of the preceding claims, characterized in that the additional nonionic hydrophilic monomer is chosen from those of formula (III), alone or as a mixture: 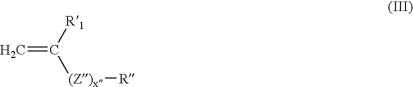
in which:
R′₁ is hydrogen or —CH₃;
Z″ is a divalent group chosen from —COO—, —CONH—, —CONCH₃—, —OCO—, —SO₂, —CO—O—CO—, —CO—CH₂—CO— and —O—; preferably COO and CONH;
x″ is 0 or 1;
R″ is a saturated or unsaturated, optionally aromatic, linear, branched or cyclic carbon-based radical of 1 to 30 carbon atoms, possibly comprising 1 to 18 heteroatoms chosen from O, N, S, F, Si and P; especially methyl, ethyl, propyl, isopropyl, n-butyl, isobutyl, tert-butyl, phenyl, benzyl or a radical of formula —CH₂—CH₂—CH₂OH, —CH₂—CH₂—OH, —CH₂—CH₂—CH₂OH or furfuryl.

23. The composition as claimed in one of the preceding claims, characterized in that the additional nonionic hydrophilic monomer is chosen from: methyl methacrylate, methyl acrylate, ethyl methacrylate, ethyl acrylate, propyl acrylate, isopropyl acrylate, tetrahydrofurfuryl methacrylate, tetrahydrofurfuryl acrylate, 2-hydroxyethyl methacrylate, 2-hydroxyethyl acrylate, ethoxyethyl methacrylate, ethoxyethyl acrylate, N-isopropylacrylamide, N-isopropylmethacrylamide, N,N-dimethylacrylamide, N,N-dimethylmethacrylamide, vinyl acetate, methyl vinyl ether, ethyl vinyl ether, vinylpyrrolidone, vinylcaprolactam, N-vinylacetamide and hydroxylpropyl acrylate; N-vinyllactam, acrylamide, N-methylacrylamide, N,N-dimethylacrylamide, N-methyl-N-vinylacetamide, N-vinylformamide, N-methyl-N-vinylformamide, vinyl alcohol (copolymerized in the form of vinyl acetate and then hydrolyzed).

24. The composition as claimed in one of the preceding claims, characterized in that the additional nonionic hydrophilic monomer, alone or as a mixture, is present in an amount of from 0.1% to 35% by weight, preferably from 1% to 25% by weight, for example from 3% to 15% by weight, or even from 5% to 9.5% by weight, relative to the total weight of the polymer.

25. The composition as claimed in one of the preceding claims, characterized in that the copolymer is neutralized.

26. The composition as claimed in one of the preceding claims, characterized in that the copolymer is neutralized with a neutralizer chosen from linear, branched or cyclic aliphatic acids and/or unsaturated or aromatic acids, and may especially contain 1 to 1000 carbon atoms and especially 2 to 500 carbon atoms; containing at least one acid function in the Brönsted sense, and especially one or more carboxylic, sulfonic and/or phosphonic acid groups; and also possibly comprising one or more heteroatoms chosen from O, N, Si, F and P, for example in the form of hydroxyl groups.

27. The composition as claimed in one of the preceding claims, characterized in that the copolymer is neutralized with a neutralizer chosen, alone or as a mixture, from:
linear, branched or cyclic, saturated or unsaturated, optionally aromatic fatty acids containing 6 to 32 and especially 8 to 28 carbon atoms, and comprising at least one COOH or sulfonic acid (—SO₃H) function;
linear, branched or cyclic, saturated or unsaturated, optionally aromatic hydroxy acids, especially α-hydroxy acids, containing 6 to 32 and especially 8 to 28 carbon atoms, and comprising at least one COOH or sulfonic acid (—SO₃H) function;
alkylbenzenesulfonic acids, in which the alkyl group may contain from 4 to 30 and especially from 6 to 24 carbon atoms;
amphoteric neutralizers, especially of the alkylbetaine or alkylamidopropylbetaine type, in which the alkyl group may contain 4 to 30 and especially 6 to 24 carbon atoms; in particular cocoamidopropylbetaine.

28. The composition as claimed in one of the preceding claims, characterized in that the copolymer is neutralized with a neutralizer chosen from α-hydroxyethanoic acid, α-hydroxyoctanoic acid, α-hydroxycaprylic acid, ascorbic acid, acetic acid, benzoic acid, behenic acid, capric acid, citric acid, caproic acid, caprylic acid, dodecylbenzenesulfonic acid, 2-ethylcaproic acid, folic acid, fumaric acid, galactaric acid, gluconic acid, glycolic acid, 2-hexadecyleicosanoic acid, hydroxycaproic acid, 12-hydroxystearic acid, isolauric acid (or 2-butyloctanoic acid), isomyristic acid (or 2-hexyloctanoic acid), isoarachidic acid (or 2-octyldodecanoic acid), isolignoceric acid (or 2-decyltetradecanoic acid), lactic acid, lauric acid, malic acid, myristic acid, oleic acid, palmitic acid, propionic acid, sebacic acid, stearic acid, tartaric acid, terephthalic acid, trimesic acid, undecylenic acid, propylbetaine, cocoamidopropylbetaine, and betaine hydrochloride of formula [(CH₃)₃N+CH₂CO₂H.Cl—], and mixtures thereof.

29. The composition as claimed in one of the preceding claims, characterized in that the copolymer is neutralized with a neutralizer chosen from caproic acid, 2-ethylcaproic acid, oleic acid, behenic acid, stearic acid, acetic acid, citric acid, tartaric acid, betaine hydrochloride and/or gluconic acid, and preferentially betaine hydrochloride and/or behenic acid.

30. The composition as claimed in one of claims 25 to 29, characterized in that the neutralizer is added in an amount of from 1% to 300% and especially from 5% to 250% or even 10% to 200% relative to the total amine functions of the polymer or of the monomers.

31. The composition as claimed in claim 30, in which the neutralizer, alone or as a mixture, is added in an amount of from 1% to 99%, especially 5% to 90% or even 10% to 80% relative to the total amine functions of the polymer or of the monomers.

32. The composition as claimed in claim 30, in which the neutralizer, alone or as a mixture, is added in an amount of from 0.01 to 3 molar equivalents, especially 0.05 to 2.5 or even 0.1 to 2 molar equivalents relative to the total amine functions of the polymer or of the monomers.

33. The composition as claimed in one of the preceding claims, characterized in that the copolymer has a weight-average molecular mass (Mw) of between 500 and 5 000 000, especially between 1000 and 3 000 000 and more preferentially between 2000 and 2 000 000, or even between 4000 and 50.0 000, better still between 7000 and 250 000 and even better between 8000 and 100 000.

34. The composition as claimed in one of the preceding claims, characterized in that the copolymer comprises:
a monomer of formula (I), alone or as a mixture, present in a proportion of from 20% to 80% by weight, especially from 20% to 60% by weight and preferably from 30% to 50% by weight, relative to the weight of the final polymer, and chosen, alone or as a mixture, from poly(ethylene glycol) (meth)acrylates, preferably those with a molecular weight of between 350 and 13 000 g/mol and especially between 500 and 8000 g/mol; and
an “essentially cationic” monomer present in a proportion of from 40% to 90% by weight, especially from 40% to 80% by weight and preferably from 50% to 70% by weight relative to the weight of the final polymer, and chosen, alone or as a mixture, from dimethylaminopropyl(meth)acrylamide, dimethylaminoethyl (meth)acrylamide, diethylaminoethyl (meth)acrylate, dimethylaminoethyl(meth)acrylate, vinylimidazole, vinylpyridine and morpholinoethyl (meth)acrylate; and
the copolymer being neutralized with a neutralizer chosen from 2-ethylcaproic acid, oleic acid, behenic acid, stearic acid, acetic acid, citric acid, tartaric acid, betaine hydrochloride and/or gluconic acid, and preferentially behenic acid and/or betaine hydrochloride.

35. The composition as claimed in one of the preceding claims, characterized in that the copolymer comprises:
a monomer of formula (I), alone or as a mixture, present in a proportion of from 20% to 80% by weight, especially from 20% to 60% by weight and preferably from 30% to 50% by weight, relative to the weight of the final polymer, and chosen, alone or as a mixture, from poly(ethylene glycol) (meth)acrylates, preferably those with a molecular weight of between 350 and 13 000 g/mol and especially between 500 and 8000 g/mol; and
an “essentially cationic”, monomer present in a proportion of from 40% to 90% by weight, especially from 40% to 80% by weight and preferably from 50% to 70% by weight, relative to the weight of the final polymer, and chosen, alone or as a mixture, from dimethylaminopropyl(meth)acrylamide, and
the polymer being neutralized with a neutralizer chosen from behenic acid and/or betaine hydrochloride.

36. The composition as claimed in one of the preceding claims, in which the copolymer is present in a proportion of from 0.01% to 30% by weight of solids, especially from 0.1% to 20% by weight or even from 0.1% to 10% by weight and better still from 0.5% to 3% by weight relative to the total weight of the composition.

37. The composition as claimed in one of the preceding claims, in which the cosmetically acceptable medium comprises at least one constituent chosen from water; hydrophilic organic solvents, for instance alcohols, especially linear or branched C₁-C₆ monoalcohols and polyols, glycol ethers, and mixtures thereof.

38. The composition as claimed in any one of the preceding claims, characterized in that the synthetic oils are polyolefins of hydrogenated or nonhydrogenated polybutene type, or of hydrogenated or nonhydrogenated polydecene type.

39. The composition as claimed in any one of claims 1 to 38, characterized in that the cationic polymers have a cationic density ranging from 0.05 to 4 meq/g.

40. The composition as claimed in any one of the preceding claims, characterized in that the cationic polymers are chosen from cylcopolymers, cationic polysaccharides and quaternary polymers of vinylpyrrolidone and of vinylimidazole, and mixtures thereof.

41. The composition as claimed in claim 40, characterized in that said cationic polysaccharides are chosen from quaternary cellulose ether derivatives and guargums modified with a 2-3-epoxypropyltrimethylammonium salt.

42. The composition as claimed in claim 40, characterized in that said quaternary cellulose ether derivatives are chosen from hydroxyethylcelluloses that have reacted with an epoxide substituted with a trimethylammonium group.

43. The composition as claimed in any one of the preceding claims, characterized in that the silicones are chosen from polyorganosiloxanes that are insoluble in the composition.

44. The composition as claimed in any one of the preceding claims, characterized in that the silicones are nonvolatile polyorganosiloxanes chosen from polyalkylsiloxanes, polyarylsiloxanes, polyalkylarylsiloxanes, silicone gums and resins, and polyorganosiloxanes modified with organofunctional groups, and mixtures thereof.

45. The composition as claimed in claim 44, characterized in that:
(a) the polyalkylsiloxanes are chosen from:
polydimethylsiloxanes with trimethylsilyl end groups;
polydimethylsiloxanes with dimethylsilanol end groups;
poly (C₁-C₂₀) alkylsiloxanes;
(b) the polyalkylarylsiloxanes are chosen from:
linear and/or branched polydimethylmethylphenylsiloxanes and polydimethyldiphenylsiloxanes with a viscosity of between 1×10⁻⁵ and 5×10⁻² m²/s at 25° C.;
(c) the silicone gums are chosen from polydiorganosiloxanes with number-average molecular masses of between 200 000 and 1 000 000, used alone or in the form of a mixture in a solvent;
(d) the resins are chosen from resins consisting of units: R₃SiO_{1/2}, R₂SiO_{2/2}, RSiO_{3/2}, SiO_{4/2}
in which R represents a hydrocarbon-based group containing from 1 to 16 carbon atoms or a phenyl group;
(e) the organomodified silicones are chosen from silicones comprising in their structure one or more organofunctional groups attached via a hydrocarbon-based radical.

46. The composition as claimed in claim 44, characterized in that the silicone gums used, alone or in the form of a mixture, are chosen from the following structures:
polydimethylsiloxane,
polydimethylsiloxane/methylvinylsiloxanes,
polydimethylsiloxane/diphenylsiloxane,
polydimethylsiloxane/phenylmethylsiloxane,
polydimethylsiloxane/diphenylsiloxane/methylvinylsiloxanes and the following mixtures:
mixtures formed from a polydimethylsiloxane hydroxylated at the end of the chain and from a cyclic polydimethylsiloxane;
mixtures formed from a polydimethylsiloxane gum and from a cyclic silicone; and
mixtures of polydimethylsiloxanes of different viscosities.

47. The composition as claimed in claim 44, characterized in that the organomodified silicones are chosen from polyorganosiloxanes comprising:
a) polyethyleneoxy and/or polypropyleneoxy groups;
b) substituted or unsubstituted amine groups;
c) thiol groups;
d) alkoxylated groups;
e) hydroxyalkyl groups;
f) acyloxyalkyl groups;
g) alkylcarboxylic groups;
h) 2-hydroxyalkylsulfonate groups;
i) 2-hydroxyalkylthiosulfonate groups;
j) hydroxyacylamino groups.

48. The composition as claimed in any one of the preceding claims, characterized in that the polyorganosiloxanes are chosen from polyalkylsiloxanes containing trimethylsilyl end groups, polyalkylsiloxanes containing dimethylsilanol end groups, polyalkylarylsiloxanes and polysiloxanes containing amine groups.

49. The composition as claimed in any one of the preceding claims, characterized in that the compounds of ceramide type are chosen from:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearbylaminooctadecane-1,3,4-triol and in particular N-stearoylphytosphingosine,
2-N-palmitoylaminohexadecane-1,3-diol
(bis(N-hydroxyethyl-N-cetyl)malonamide),
cetylic acid N-(2-hydroxyethyl)-N-(3-cetyloxy-2-hydroxypropyl)amide,
N-docosanoyl-N-methyl-D-glucamine
or mixtures of these compounds.

50. The composition as claimed in any one of the preceding claims, characterized in that the conditioning agent(s) is (are) present in a concentration ranging from 0.001% to 20% by weight and preferably from 0.01% to 10% by weight relative to the total weight of the composition.

51. The composition as claimed in any one of the preceding claims, characterized in that the composition also comprises at least one additive chosen from fatty alcohols containing from 12 to 26 carbon atoms; polymers; vitamins, fragrances, nacres, thickeners, gelling agents, trace elements, softeners, sequestrants, fragrances, acidifying or basifying agents, preserving agents, sunscreens, antioxidants, hair-loss counteractants, antidandruff agents, propellants and ceramides; mixtures thereof.

52. The composition as claimed in any one of the preceding claims, characterized in that it also comprises at least one surfactant chosen from anionic, nonionic and amphoteric surfactants, and mixtures thereof.

53. The composition as claimed in claim 52, characterized in that the additional surfactant(s) is (are) present in a concentration of between 0.1% and 60% by weight and preferably between 1% and 40% by weight.

54. The composition as claimed in one of the preceding claims, which is in the form of a haircare composition, especially for holding the hair style or shaping the hair, for example in the form of shampoos, hairsetting gels or lotions, blow-drying lotions and fixing and styling compositions such as lacquers or sprays; rinse-out or leave-in hair conditioners, compositions for permanent-waving, relaxing, dyeing or bleaching the hair, or alternatively in the form of rinse-out compositions, to be applied before or after dyeing, bleaching, permanent-waving or relaxing the hair or alternatively between the two steps of a permanent-waving or hair-relaxing operation.

55. A cosmetic process for treating keratin materials such as bodily or facial skin, the nails, bodily hair, head hair and/or the eyelashes, characterized in that it consists in applying to the keratin materials a cosmetic composition as defined in one of claims 1 to 46 and in optionally following the application by rinsing, after an optional action time.

56. The use of a composition as defined in any one of the preceding claims, for washing or caring for keratin materials.

57. The use of a composition as defined in any one of the preceding claims, for holding or shaping keratin materials.
